(19) United States
(12) Patent Application Publication
(10) Pub. No.: US 2025/0367170 A1
(43) Pub. Date: Dec. 4, 2025

(54) TOPICAL ANGIOTENSIN II RECEPTOR BLOCKERS (ARBS) FOR TREATING EYE CONDITIONS

(71) Applicant: The Cleveland Clinic Foundation, Cleveland, OH (US)

(72) Inventor: Steven E. WILSON, Cleveland, OH (US)

(21) Appl. No.: 18/868,381

(22) PCT Filed: Dec. 22, 2022
(86) PCT No.: PCT/US2022/082201

§ 371 Date: Nov. 22, 2024

Related U.S. Application Data
(60) Provisional application No. 63/345,202, filed on May 24, 2022.
(60) Provisional application No. 63/392,917, filed on Jul. 28, 2022.
(60) Provisional application No. 63/375,949, filed on Sep. 16, 2022.
(60) Provisional application No. 63/385,138, filed on Nov. 28, 2022.

(51) Int. Cl. A61K 31/4178; A61K 9/00; A61K 9/06; A61K 45/06; A61P 27/02

(52) U.S. Cl. CPC A61K 31/4178; A61K 9/0048; A61K 9/06; A61K 45/06; A61P 27/02

(57) ABSTRACT

The present invention relates to compositions, systems, and methods for treating a subject with an eye condition (e.g., topically) using a composition comprising an ACE-2 receptor antagonist (also known as Angiotensin II Receptor Blocker or “ARB”) (e.g., losartan, telmisartan, valsartan, olmesartan, candesartan, irbesartan, eprosartan, azilsartan, or losartan metabolite EXP3174). In certain embodiments, the eye condition is selected from: i) a corneal scarring fibrosis, ii) a transforming growth factor beta-induced (TGFBI) corneal dystrophy, iii) a conjunctival fibrotic disease, iv) an intraocular fibrotic disease, and v) a conjunctival bleb scarring and/or shunt encapsulation (e.g., following glaucoma surgery).

The present application claims priority to the following U.S. Provisional applications, 63/345,202, filed May 24, 2022; 63/392,917 filed Jul. 28, 2022; 63/375,949 filed Sep. 16, 2022; and 63/385,138 filed Nov. 28, 2022; all of which are herein incorporated by reference in their entireties:

### STATEMENT REGARDING FEDERAL FUNDING

This invention was made with government support under W81XWH-19-1-0846 awarded by the Department of Defense (CDMRP). The government has certain rights in the invention.

### SEQUENCE LISTING

The text of the computer readable sequence listing filed herewith, titled “41314-601_SEQUENCE_LISTING”, created Dec. 22, 2022, having a file size of 1,936 bytes, is hereby incorporated by reference in its entirety.

### FIELD OF THE INVENTION

The present invention relates to compositions, systems, and methods for treating a subject with an eye condition (e.g., topically) using a composition comprising an ACE-2 receptor antagonist (also known as Angiotensin II Receptor Blocker or “ARB”) (e.g., losartan, telmisartan, valsartan, olmesartan, candesartan, irbesartan, eprosartan, azilsartan, or losartan metabolite EXP3174). In certain embodiments, the eye condition is selected from: i) a corneal scarring fibrosis, ii) a transforming growth factor beta-induced (TGFBI) corneal dystrophy, iii) a conjunctival fibrotic disease, iv) an intraocular fibrotic disease, and v) a conjunctival bleb scarring and/or shunt encapsulation (e.g., following glaucoma surgery).

### BACKGROUND OF THE INVENTION

Corneal scarring fibrosis mediated by the development of myofibroblasts after traumatic injury, microbial infections, scarring diseases and some corneal surgeries is one of the most important causes of vision loss in the U.S.A. and throughout the world (Witcher et al., 2001). According to WHO statistics 5.1% of bilateral blindness is corneal blindness and stromal scarring is the largest subcategory (Witcher et al., 2001). Corneal opacity due to microbial keratitis or trauma is also a common reason for corneal transplantation in the U.S.A. (Ghosheh et al., 2008). Persistent corneal scarring due to trauma, infection, disease, or surgeries occurs by the same myofibroblast-related mechanisms in humans (Cockerham and Hidayat, 1999; Lee et al., 2001), as it does in rabbits, mice, rats, chickens, and other species (Mohan et al., 2003; Netto et al., 2006; Martinez-Garcia, et al., 2006; Mohan et al., 2008; Hindman et al., 2019; Joung et al., 2020; de Oliveira et al., 2021).

### SUMMARY OF THE INVENTION

The present invention relates to compositions, systems, and methods for treating a subject with an eye condition (e.g., topically) using a composition comprising an ACE-2 receptor antagonist (also known as an Angiotensin II Receptor Blocker) (e.g., losartan, telmisartan, valsartan, olmesartan, candesartan, irbesartan, eprosartan, azilsartan, or losartan metabolite EXP3174). In certain embodiments, the eye condition is selected from: i) a corneal scarring fibrosis, ii) a transforming growth factor beta-induced (TGFBI) corneal dystrophy, iii) a conjunctival fibrotic disease, iv) an intraocular fibrotic disease, and v) a conjunctival bleb scarring and/or shunt encapsulation (e.g., following glaucoma surgery). In particular conditions, the eye condition comprises keratoconus.

In certain embodiments, the ACE-2 receptor antagonists (aka Angiotensin II Receptor Blockers or ARBs) is present in the composition at a concentration of about 0.01 to 0.9 mg/ml or 0.05 mg/ml to 0.9 mg/ml or about 0.1 mg/ml to 3.0 mg/ml or about 0.01 to 0.4 mg/ml (e.g., 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 . . . 2.5 . . . or 3.0 mg/ml). In some embodiments, the ACE-2 receptor antagonists (aka ARBs) present in the composition at a concentration of about 0.01 to 400 mg/ml (e.g., 0.01 . . . 10 . . . 50 . . . 100 . . . 200 . . . 300 . . . or 400 mg/ml). In certain embodiments, the ACE-2 receptor is present in the composition at a concentration of 0.01-0.09 or 2.3-100 mg/ml (e.g., 2.3 . . . 8.0 . . . 15 . . . 25 . . . 75 . . . or 100 mg/ml).

In some embodiments, provided herein are compositions comprising: a) a drug agent, wherein the drug agent comprises an ACE-2 receptor antagonist (e.g., losartan) which is present in said composition at a concentration of 0.01-0.09 or 2.3-100 mg/ml, b) water, and c) at least one of the following: i) one or more salts present at a level such that the composition, when in aqueous form, has about a physiological concentration of the one or more salts and about a physiological pH; ii) one or more gelling agents present at a level such that the composition is in the form of a gel; and iii) one or more ointment forming agents, present at a level such that the composition is in the form of an ointment; d) optionally a preservative (e.g., boric acid), and e) optionally a soothing agent.

In certain embodiments, provided herein are methods comprising: delivering a system to a subject, wherein the subject has an eye that comprises a corneal injury or an existing corneal scar, and wherein the system comprises: a) an eye dropper container or a contact lens, and b) a composition comprising: i) a drug agent, wherein the drug agent comprises an ACE-2 receptor antagonist (aka Angiotensin II Receptor Blocker) (e.g., losartan), ii) water, and iii) at least one of the following: A) one or more salts present at a level such that the composition, when in aqueous form, has about a physiological concentration of the one or more salts and about a physiological pH, B) one or more gelling agents present at a level such that the composition is in the form of a gel; and C) one or more ointment forming agents, present at a level such that the composition is in the form of an ointment; iv) optionally a preservative (e.g., boric acid), and v) optionally a soothing agent.

In further embodiments, provided herein are systems comprising: a) composition comprising: i) a drug agent, wherein the drug agent comprises an ACE-2 receptor antagonist (aka Angiotensin II Receptor Blocker) (e.g., losartan), wherein said ACE-2 receptor antagonist is present in said composition at a concentration of 0.01-0.09 or 2.3-100 mg/ml, ii) water, and iii) at least one of the following: A) one or more salts present at a level such that the composition, when in aqueous form, has about a physiological concentration of the one or more salts and about a physiological pH; B) one or more gelling agents present at a level such that the composition is in the form of a gel; and C) one or more ointment forming agents, present at a level such that the composition is in the form of an ointment; iv) optionally a preservative (e.g., boric acid), and v) optionally a soothing agent; and b) an eye dropper container or a contact lens. In particular embodiments, they system comprises the eye dropper, and wherein the composition is present inside the eye dropper. In further embodiments, the system comprises the contact lens, and wherein the composition is present inside of, or on the inner surface of, the contact lens.

In some embodiments, provided herein are methods of treating a subject with a corneal injury and/or an existing corneal scar comprising: administering a composition (e.g., topically) to a cornea of a subject, or providing the composition to the subject such that the subject administers the composition to the cornea, wherein the cornea of the subject comprises a corneal injury and/or an existing corneal scar, and wherein the composition comprises: a) a drug agent, wherein the drug agent comprises an ACE-2 receptor antagonist (aka Angiotensin II Receptor Blocker) (e.g., losartan), b) water, and c) at least one of the following: i) one or more salts present at a level such that the composition, when in aqueous form, has about a physiological concentration of the one or more salts and about a physiological pH; ii) one or more gelling agents present at a level such that the composition is in the form of a gel; and iii) one or more ointment forming agents, present at a level such that the composition is in the form of an ointment; d) optionally a preservative (e.g. boric acid), and e) optionally a soothing agent.

In certain embodiments, provided herein are methods of treating a subject with an eye condition comprising: administering a composition to a cornea and/or conjunctiva of a subject, or providing the composition to the subject such that the subject administers the composition to the cornea and/or conjunctiva, wherein the cornea and/or conjunctiva of the subject comprises an eye condition, and wherein the composition comprises: a) a drug agent, wherein the drug agent comprises an ACE-2 receptor antagonist (aka Angiotensin II Receptor Blocker), and wherein the eye condition is selected from: i) a corneal scarring fibrosis, ii) a TGFBI corneal dystrophy, iii) a conjunctival fibrotic disease, iv) an intraocular fibrotic disease, and v) a conjunctival bleb scarring and/or shunt encapsulation (e.g., following glaucoma surgery or other surgery).

In certain embodiments, provided herein are methods comprising: delivering a system to a subject with an eye condition, wherein the eye condition is selected from: i) a corneal scarring fibrosis, ii) a TGFBI corneal dystrophy, iii) a conjunctival fibrotic disease, iv) an intraocular fibrotic disease, and v) a conjunctival bleb scarring and/or shunt encapsulation (e.g., following glaucoma surgery), and wherein the system comprises: a) an eye dropper container or a contact lens, and b) a composition comprising: i) a drug agent, wherein the drug agent comprises an ACE-2 receptor antagonist (aka Angiotensin II Receptor Blocker).

In further embodiments, the corneal scarring fibrosis is selected from the group consisting of: Descemetorhexis without graft, DSAEK or DMEK graft displacement, Alkali or acid burns, sulfur mustard, chemical weapon burn, proliferative vitreoretinopathy (PVR), PRK late haze, PRK breakthrough late haze after MMC treatment, LASIK complications scar, persistent epithelial defect, recurrent Herpes simplex virus (HSV) keratitis, HSV endotheliitis with scarring, Herpes zoster ophthalmicus, bacterial keratitis, fungal keratitis, and/or acanthamoeba keratitis, Corneal lacerations with excessive fibrosis. In other embodiments, the TGFBI corneal dystrophy is selected from the group consisting of: a stromal deposit, a Reis-Bucklers corneal dystrophy, a Thiel-Behnke corneal dystrophy, a Lattice corneal dystrophy type 1, a Granular corneal dystrophy type 1 and/or type 2. In additional embodiments, the Conjunctival fibrotic disease is selected from the group consisting of: Trachoma, Stevens-Johnson syndrome, ocular cicatricial pemphigoid, and ocular graft-vs-host disease. In some embodiments, the intraocular fibrotic disease is proliferative vitreoretinopathy.

In particular embodiments, wherein the drug agent (Angiotensin II Receptor Blocker or ACE-2 receptor antagonist) is present in the composition at a concentration of 0.1 mg/ml to 2.0 mg/ml or about 0.05 mg/ml to about 3.0 mg/ml or about 0.01 to about 0.9 mg/ml or about 0.01 to about 400 mg/ml (e.g., 0.1, 0.3, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.8, 0.9, 3.0 . . . 50 . . . 75 . . . 100 . . . 125 . . . 200 . . . 250 . . . 350 . . . 400 mg/ml). In other embodiments, the administering, or the administers, is conducted at least daily for at least one week, or at least 2 weeks, or at least one month, or at least a year, and wherein the subject has a best corrected visual acuity (BSCVA) that is 20/X just prior to the administering or the administers, and is 20/Y at then end of the at least one week, the at least 2 weeks, or the at least one month, or the at least one year, and wherein Y is at least 5 points (or 10, or 15, or 20, or 25, or 30 points) lower than X.

In certain embodiments, the composition is free, or detectably free, of any additional reagents besides the drug agent (Angiotensin II Receptor Blocker, aka ACE-2 receptor antagonist), the water, and the one or more salts. In other embodiments, the composition comprises the soothing agent, and wherein the composition is free, or detectably free, of any additional reagents besides the drug agent, the water, the one or more salts, and the soothing agent. In some embodiments, the composition further comprises the preservative, and wherein the composition is free, or detectably free, of any additional reagents besides the drug agent, the water, the one or more salts, and the preservative. In certain embodiments, the composition further comprises the preservative and the soothing agent, and wherein the composition is free, or detectably free, of any additional reagents besides the drug agent, the water, the one or more salts, the preservative, and the soothing agent.

In particular embodiments, the preservative is selected from the group consisting of: benzalkonium chloride, sodium chlorite, sodium perborate, purite, benzododecinium bromide, ethylenediaminetetraacetic acid (EDTA), chlorobutanol, thiomersal, disodium edetate, oxychloro complex (SOC), and boric acid. In certain embodiments, the soothing agent is present in the composition, and wherein the soothing agent is optionally selected from the group consisting of: carboxymethyl cellulose, polyvinyl alcohol, hydroxypropyl methylcellulose, hydroxypropyl cellulose, and hyaluronic acid.

In some embodiments, the composition is present in an eyedrop container, and optionally wherein the eyedrop container is a single-use container. In further embodiments, the composition comprises the one or more salts and is in a liquid form, and further is free or detectably free of the one or more gelling agents and the one or more ointment forming agents. In additional embodiments, the composition comprises the one or more gelling agents and/or the one or more ointment forming agents, and is in the form of a gel or an ointment, and wherein optionally the gelling agents are selected from the group consisting of: hypromellose (e.g., about 0.3%), carbomer homopolymer (e.g., about 0.5%), and carboxymethylcellulose (e.g., about 1%), and wherein optionally the ointment forming agent is mineral oil (e.g., about 40-50%) and/or petrolatum (e.g., 40-60%).

In some embodiments, the administering, or the administers, is at least four or six or eight times daily for at least one week. In further embodiments, the administering, or the administers, is conducted about every half hour for at least 8 hours. In additional embodiments, the cornea of the subject comprises the corneal injury, and the administering, or the administers, is conducted at least daily for at least one week beginning no more than 1-5 days from the occurrence of the corneal injury, wherein after one month from the occurrence of the corneal injury, the cornea has a Fantes slit-lamp corneal haze score of 0, 0.5, 1, or 2, wherein the corneal injury would have produced a Fantes slit-lamp corneal haze score of 3 or 4 after the one month if left untreated. In other embodiments, the cornea of the subject comprises the corneal injury, and the administering, or the administers, is conducted at least daily for at least one week beginning no more than 1-5 days from the occurrence of the corneal injury, wherein after one month from the occurrence of the corneal injury, the cornea has a Fantes slit-lamp corneal haze score of 0, 0.5, or 1, wherein the corneal injury would have produced a Fantes slit-lamp corneal haze score of 2, 3, or 4 after the one month if left untreated.

In particular embodiments, the drug agent is selected from the group consisting of: losartan, telmisartan, valsartan, olmesartan, candesartan, irbesartan, eprosartan, azilsartan, and losartan metabolite EXP3174. In other embodiments, the one or more salts comprise one or more, or all, of the following: i) about 0.64%, or 0.60%-0.070%, sodium chloride, ii) about 0.075%, or 0.070-0.080%, potassium chloride, iii) about 0.048%, or 0.040-0.055%, calcium chloride dihydrate, iv) about 0.03%, or 0.01-0.05%, magnesium chloride hexahydrate, v) about 0.39%, or 0.30-0.50, sodium acetate trihydrate, and/or vi) about 0.17%, or about 0.10-0.30%, sodium citrate dihydrate.

In certain embodiments, the methods further comprise: administering a corticosteroid to the cornea of the subject, or providing the corticosteroid to the subject such that the subject administers the corticosteroid to the cornea, wherein the corticosteroid is present in the composition or present in a separate composition. In other embodiments, the composition is present in a conjunctival reservoir, or other continuous delivery device, that slowly releases the composition over time into the tears of the subject or slowly releases the composition over time intraocular in the subject (e.g., for treating PVR (proliferative vitreoretinopathy)). In certain embodiments, the composition is present in a porous collagen therapeutic contact lens that releases the composition over time. In certain embodiments, the devices and compositions employed for slow release over time in tear or intraocular are as described in, for example, Bourges, et al., Advanced Drug Delivery Reviews 58 (2006) 1182-1202, which is herein incorporated by reference and in particular for the slow release devices and compositions described therein.

In some embodiments, the composition comprises the preservative (e.g., an antibiotic). In further embodiments, the administering, or the administers, is conducted at least daily for at least one week, or at least 2 weeks, or at least one month, and wherein the subject has myopia score of X diopters just prior to the administering or the administers, and is Y diopters at the end of the at least one week, the at least 2 weeks, or the at least one month, and wherein Y is at least 1 diopter lower than X. In particular embodiments, the subject is a human, cat, dog, horse, cow, pig, or other vertebrate animal.

In particular embodiments, the cornea of the subject comprises the corneal injury, and wherein the corneal injury has occurred 1, 3, 6, 12, 24, or 48 hours prior to the administering or the administers. In other embodiments, the administering a composition to a cornea of a subject comprises the subject administering the composition to their own cornea. In other embodiments, the cornea of the subject comprises the corneal injury, and wherein the injury was caused by trauma, a chemical burn, a microbial infection, or a surgery. In particular embodiments, the cornea of the subject comprises the corneal injury, and wherein the injury was caused by photorefractive keratectomy (PRK) or phototherapeutic keratectomy (PTK). In some embodiments, the subject has Trachoma.

In certain embodiments, the cornea injury has occurred within five or less days of the administering or the administers. In further embodiments, the cornea injury has occurred within 24 hours or less of the administering or the administers. In additional embodiments, the drug agent comprises losartan. In certain embodiments, the composition is present in the eye dropper container. In other embodiments, the composition is present in the contact lens. In some embodiments, a subject with a corneal injury or corneal scar is treated by providing contact lenses impregnated with the compositions herein (e.g., losartan) to the subject which they install, or installing such contact lenses in the subject's eyes for them.

In some embodiments, provided herein are methods of treating a subject with a corneal injury (e.g., occurring recently) and/or an existing corneal scar (e.g., one that has existed for three, four, five, six months or more) comprising: administering a first composition (e.g., topically) to a cornea of a subject, or providing said composition to said subject such that said subject administers said composition to said cornea, wherein said cornea of said subject comprises a corneal injury and/or an existing corneal scar, and wherein said composition comprises a drug agent, wherein said drug agent comprises an ACE-2 receptor antagonist (aka Angiotensin II Receptor Blocker); wherein optionally said drug agent is selected from the group consisting of: losartan, telmisartan, valsartan, olmesartan, candesartan, irbesartan, eprosartan, azilsartan, losartan metabolite EXP3174, and optionally administering a corticosteroid to the cornea of the subject, or providing the corticosteroid to the subject such that the subject administers the corticosteroid to the cornea, wherein the corticosteroid is present in the first composition or in a second composition. In some embodiments, the first and/or second compositions are in the form of an oil-in-water emulsion or micelle. In certain embodiments, the ACE-2 receptor antagonist (aka Angiotensin II Receptor Blocker) is present in the first and/or second compositions at 0.001-30 mg/ml or 0.1-30 mg/ml or 0.05-30 mg/ml, such as 0.001 . . . 0.05 . . . 1.0 . . . 5.0 . . . 10.0 . . . 15.0 . . . 20.0 . . . 25.0 . . . or 30.0 mg/ml . . . 70 . . . 125 . . . 150 . . . 300 . . . or 400 mg/ml (e.g., if present in an oil-in-water emulsion or micelle).

In particular embodiments, provided herein are methods comprising: delivering a system to a subject, wherein said subject has an eye that comprises a corneal injury or an existing corneal scar, and wherein said system comprises: a) an eye dropper container or a contact lens, and b) a first composition comprising a drug agent, wherein said drug agent comprises an ACE-2 receptor antagonist (aka Angiotensin II Receptor Blocker), wherein said drug agent is optionally selected from the group consisting of: losartan, telmisartan, valsartan, olmesartan, candesartan, irbesartan, eprosartan, azilsartan, losartan metabolite EXP3174, and c) optionally a corticosteroid, wherein said corticosteroid is present in said first composition or in a second composition. In certain embodiments, the drug agent is present in the composition at a concentration of 0.01 mg/ml-5.0 mg/ml or 0.05 mg/ml to 0.9 mg/ml, or 0.1 mg/ml to 0.9 mg/ml, or about 0.05 mg/ml to 3.0 mg/ml. In certain embodiments, the corticosteroid is employed in the method and is present in the first composition. In other embodiments, the corticosteroid is employed in the method and is present in the second composition. In some embodiments, the first and/or second compositions are in the form of an oil-in-water emulsion or micelle. In certain embodiments, the ACE-2 receptor antagonist is present in the first and/or second compositions at 0.01-30 mg/ml or 0.05-30 mg/ml, such as 0.01 . . . 0.05 . . . 1.0 . . . 5.0 . . . 10.0 . . . 15.0 . . . 20.0 . . . 25.0 . . . or 30.0 mg/ml . . . or up to over 400 mg/ml (e.g., if present in an oil-in-water emulsion or micelle).

In some embodiments, the delivery, or administering, (e.g., of the first and/or second composition) is performed by a pharmacy employee, a doctor, a nurse, or other healthcare worker. In certain embodiments, the corneal injury has occurred within five or less days of the delivering or administering of first and/or second composition (e.g., 5, 4, 3, 2, or 1 day). In other embodiments, the corneal injury has occurred within 24 hours or less of the delivering of the first and/or second composition (e.g., 24 . . . 12 . . . 6 . . . 3 . . . 2 . . . or 1 hour).

In some embodiments, provided herein are compositions comprising, or consisting essentially of, a drug agent and saline solution, and optionally a corticosteroid, wherein said drug agent comprises an ACE-2 receptor antagonist (aka Angiotensin II Receptor Blocker), wherein said drug agent is optionally selected from the group consisting of: losartan, telmisartan, valsartan, olmesartan, candesartan, irbesartan, eprosartan, azilsartan, losartan metabolite EXP3174.

In particular embodiments, provided herein are systems comprising: a) a first composition comprising: comprising a drug agent, wherein said drug agent comprises an ACE-2 receptor antagonist (aka Angiotensin II Receptor Blocker), wherein said drug agent is optionally selected from the group consisting of: losartan, telmisartan, valsartan, olmesartan, candesartan, irbesartan, eprosartan, azilsartan, losartan metabolite EXP3174, and b) an eye dropper container or a contact lens, and c) optionally a corticosteroid, wherein said corticosteroid is present in said first composition or in a second composition. In particular embodiments, the system comprises the eye dropper, and wherein the first and/or second composition is present inside the eye dropper. In further embodiments, the system comprises the contact lens, and wherein the first and/or second composition is present inside of, or on the inner surface of, the contact lens.

In particular embodiments, the compositions herein are sterile. In some embodiments, the drug agent is present in the first composition at a concentration of about 0.01-0.9 mg/ml or 0.05-0.9 mg/ml or about 0.05 mg/ml to 2.0 mg/ml. In further embodiments, the concentration of 0.05-0.9 mg/ml of the drug agent in the first composition is about 0.8 mg/ml, or about 0.7 mg/ml, or about 0.6 mg/ml, or about 0.5 mg/ml, or about 0.4 mg/ml, or about 0.3 mg/ml or about 0.2 mg/ml, or about 0.1 mg/ml, or about 0.05 mg/ml or about 0.01 mg/ml. In particular embodiments, the first and/or second composition is in the form of a liquid or gel and further comprises saline solution. In additional embodiments, the first composition consists of, or consists essentially of, the drug agent and the saline solution; and/or wherein the composition has a pH of 7.0 to 7.2. In some embodiments, the first and/or second compositions are in the form of an oil-in-water emulsion or micelle. In certain embodiments, the ACE-2 receptor antagonist (aka Angiotensin II Receptor Blocker) is present in the first and/or second compositions at 0.01-0.9 mg/ml or 0.05-30 mg/ml, such as 0.01 . . . 0.05 . . . 0.10 . . . 0.02 . . . 0.05 . . . 1.0 . . . 5.0 . . . 10.0 . . . 15.0 . . . 20.0 . . . 25.0 . . . or 30.0 mg/ml . . . or up to over 400 mg/ml (e.g., if present in an oil-in-water emulsion or micelle).

In certain embodiments, the first and/or second composition further comprises a preservative. In some embodiments, the preservative is selected from the group consisting of: benzalkonium chloride, sodium chlorite, sodium perborate, purite or benzododecinium bromide. In additional embodiments, the first and/or second composition is preservative-free.

In some embodiments, the first and/or second composition is present in an eyedrop container. In other embodiments, the eyedrop container is a single-use container.

In certain embodiments, the administering, or the administers, (e.g., for the ACE-2 receptor antagonist and/or the corticosteroid) is at least four times daily (e.g., 4, 5, 6, 7, 8, 9, 10, 11, 12, 13, 14, 15, 16, 17, 18, 19, 20, 21, 22, 23, or 24 times daily) for at least one week (e.g., at least 1, 2, 3, 4, 5, 6, 7, or 8 weeks). In other embodiments, the administering, or the administers, (e.g., for the ACE-2 receptor antagonist and/or the corticosteroid) is at least eight times daily for at least one week. In certain embodiments, the administering, or the administers, (e.g., for the ACE-2 receptor antagonist and/or the corticosteroid) is conducted about every half hour for at least 8 hours (e.g., at least 8, 9, 10, 11, 12, 13, 14, 15, 16, 17, 18, 19, 20 hours, . . . or 72 hours, or up to 3 days).

In some embodiments, the cornea of the subject comprises the corneal injury, and the administering, or the administers, (e.g., for the ACE-2 receptor antagonist and/or the corticosteroid) is conducted at least daily for at least one week beginning no more than 1-5 days from the occurrence of the corneal injury, wherein after one month from the occurrence of the corneal injury, the cornea has a Fantes slit-lamp corneal haze score of 0, 0.5, 1, or 2, wherein the corneal injury would have produced a Fantes slit-lamp corneal haze score of 3 or 4 after the one month if left untreated. In other embodiments, the cornea of the subject comprises the corneal injury, and the administering, or the administers, (e.g., for the ACE-2 receptor antagonist and/or the corticosteroid) is conducted at least daily for at least one week beginning no more than 1-5 days from the occurrence of the corneal injury, wherein after one month from the occurrence of the corneal injury, the cornea has a Fantes slit-lamp corneal haze score of 0, 0.5, or 1, wherein the corneal injury would have produced a Fantes slit-lamp corneal haze score of 2, 3, or 4 after the one month if left untreated. In additional embodiments, the cornea of the subject comprises the corneal injury, and the administering, or the administers, (e.g., for the ACE-2 receptor antagonist and/or the corticosteroid) is conducted at least daily for at least one week beginning no more than 1-5 days from the occurrence of the corneal injury, wherein after one month from the occurrence of the corneal injury, the cornea has a Fantes slit-lamp corneal haze score of 0 or 0.5, 1, wherein the corneal injury would have produced a Fantes slit-lamp corneal haze score of 1, 2, 3, or 4 after the one month if left untreated. In some embodiments, the cornea of the subject comprises the corneal injury, and the administering, or the administers, (e.g., for the ACE-2 receptor antagonist and/or the corticosteroid) is conducted at least daily for at least one week beginning no more than 1-5 days from the occurrence of the corneal injury, wherein after one month from the occurrence of the corneal injury, the cornea has a Fantes slit-lamp corneal haze score of 0, 0.5, 1, 2, or 3 wherein the corneal injury would have produced a Fantes slit-lamp corneal haze score of 4 after the one month if left untreated.

In particular embodiments, the first and/or second composition is in the form of an ointment, liquid, or gel. In other embodiments, the first and/or second composition is present in a conjunctival reservoir, or other continuous delivery device, that slowly releases the ACE-2 receptor antagonist (aka Angiotensin II Receptor Blocker) (e.g., losartan) and/or corticosteroid, over time into the tears of the subject. In other embodiments, the first and/or second composition is present in a porous collagen therapeutic contact lens that releases the ACE-2 receptor antagonist (e.g., losartan) or corticosteroid over time. In additional embodiments, the first and/or second composition further comprises an antibiotic. In particular embodiments, the antibiotic is selected from the group consisting of: ciprofloxacin, ofloxacin, gatifloxacin, levofloxacin, moxifloxacin, besifloxacin, gentamycin, tobramycin, amikacin, neomycin, amphotericin B, natamycin, chlorhexidine digluconate, polyhexamethyline biguanide, or brolene. In certain embodiments, the Ace-2 receptor antagonist containing compositions include at least one anti-viral agent (e.g., for herpes simplex virus, such as acyclovir, valcyclovir, famciclovir, ganciclovir, and trifluridine), and/or include anti-acanthamoeba drugs, such as propamidine isethionate, hexamidine and pentamidine.

In certain embodiments, the subject is a human subject. In additional embodiments, the cornea of the subject comprises the corneal injury, and wherein the corneal injury has occurred 1, 3, 6, 12, 24, or 48 hours prior to the administering or the administers of the first and/or second composition. In some embodiments, the cornea of the subject comprises the corneal scar, and the administering, or the administers, (of the first and/or second composition) is conducted at least daily for at least one week such that the Fantes slit-lamp corneal haze score of the cornea is reduced by at least 0.5 or at least 1 from the initial Fantes slit-lamp corneal haze score of the cornea. In some embodiments, the cornea of the subject comprises the corneal injury, and wherein the injury was caused by trauma, a chemical burn, a microbial infection (e.g., chlamydia, or a surgery. In other embodiments, the cornea of the subject comprises the corneal injury, and wherein the injury was caused by photorefractive keratectomy or phototherapeutic keratectomy. In further embodiments, the cornea injury has occurred within five or less days of the administering or the administers or the first and/or second composition. In other embodiments, the cornea injury has occurred within 24 hours or less of the administering or the administers of the first and/or second composition. In some embodiments, the drug agent comprises losartan. In particular embodiments, the drug agent comprises losartan metabolite EXP3174. In certain embodiments, the eye disease or condition treated comprises keratoconus.

In certain embodiments, the compositions herein further comprise one or more of the following reagents: methylcellulose (e.g., 0.5%, 1%, 1.5% or 2% or any concentration from 0.5% to 2%); hydroxypropyl methylcellulose (e.g., 0.5%, 1%, 1.5% or 2% or any concentration from 0.5% to 2%); dextran (e.g., 0.1% or 0.2%); glycerin (e.g., 0.1% to 2%); Carbomer (e.g., 0.1% to 0.5%); hyaluronic acid (e.g., to increase corneal penetration and improve patient comfort; with molecular weights varying from about 360 to about 1200 kDa; present at 0.03%, 0.05%, 0.1%, 0.2%, 0.3%, 0.4%, 0.5%, 1% or any concentration from 0.03% to 1%); and higher viscosity lipids that may increase corneal penetration and improve patient comfort such as phospholipids, saturated and unsaturated fatty acids or triglycerides (e.g., 0.5% to 5%);

In some embodiments, the compositions herein further comprise one or more of the following reagents: ingredients that increase corneal epithelial permeability to increase losartan or other drug agents herein penetration into the stroma for greater TGF beta blockade. Such reagents could be used, for example, it would typically be used for about 1 to 5 days after infection or injury for greater blockade of TGF beta in the early phases of the scarring response. Added ingredients to accomplish this, include, for example benzalkonium chloride (e.g., 0.02%), and sodium ethylenediaminetetraacetic acid (e.g., 0.01%).

In other embodiments, the compositions herein further include an anti-inflammatory agent (e.g., used 1 day to 2 weeks after injury), such as a corticosteroid. In certain embodiments, the anti-inflammatory agent is selected from: Prednisolone acetate (e.g., 0.1%, 0.2%, 0.5%, 1% or any concentration 0.1% to 1%); fluromethalone (e.g., 0.1%, 0.25%, 0.5% or any concentration 0.1% to 0.5%); dexamethasone sodium phosphate (e.g., 0.1% to 0.2%); loteprednol (e.g., 0.1% to 1%); and difluprednate (e.g., 0.01% to 0.1%).

### DESCRIPTION OF THE DRAWINGS

FIG. 1. Standardized slit lamp photographs of representative unwounded sham surgery corneas and corneas at one month after an 8 mm central Descemetorhexis treated with topical and/or oral losartan solutions or corresponding vehicle solutions. Sham unwounded control (Con) corneas treated with oral losartan (A) or topical losartan (B). C. Cornea that had Descemetorhexis and treatment with oral and topical vehicles for one month. Arrowheads indicate neovascularization. D. Cornea that had Descemetorhexis and treatment with 5 mg/kg oral losartan three times a day. Arrowheads indicate neovascularization. E. Cornea that had Descemetorhexis and treatment with 0.4 mg/ml topical losartan six times a day for one month. Note the transparency of the peripheral cornea compared to C and D, along with a decrease in central opacity and an area of clearing (arrow), as well as a decrease in corneal neovascularization compared to C or D. F. Cornea that had Descemetorhexis and treatment with both 0.1 mg/ml topical losartan six times a day and 5 mg/kg oral losartan three times a day also had greater transparency of the peripheral cornea compared to C and D and a decrease in central opacity. An area of peripheral clearing of opacity was also present in this cornea (arrow). G. Graph showing intensity of opacity measured with ImageJ in a 2.5 mm diameter circle for each cornea. Means and standard errors for each group are shown. * and ** indicate that the intensity of opacity for DMR+topical losartan and DMR+topical and oral losartan, respectively, were significantly different from the other groups, but were not significantly different from each other. DMR is Descemetorhexis. Un is sham unwounded. OR is oral. T or TOP is topical. L or Los is losartan.

FIG. 2A. Duplex IHC for α-smooth muscle actin (SMA) marker for myofibroblasts and keratocan marker for keratocytes in corneas at one month after treatment with losartan or vehicles. Representative central sections from two corneas in each group are shown. Note the thickness of the fibrotic SMA-positive (red) layer was greater in corneas in the DMR oral vehicle and topical vehicle group (DMR vehicle, panels A, B) and DMR oral losartan group (panels E, F) compared to corneas in the DMR topical losartan group (panels C, D) and DMR topical losartan and oral losartan group (G, H). SMA-positive cells noted at the limbus in some corneas are pericytes associated with limbal blood vessels. The DMR topical losartan cornea shown in panel D had the least posterior SMA-positive fibrosis of any cornea in that group. Note there was no posterior corneal fibrosis in uninjured sham surgery corneas treated with oral (panels I, J) or topical (panels K, L) losartan. Blue is DAPI staining of cell nuclei.

FIG. 2B provides graphic results of the individual SMA-positive fibrosis area in each cornea for each group. Individual measurements and the mean+/−standard error for each group are shown. See Table 4 for Kruskal-Wallis test followed by post-hoc Dunn's-Bonferroni test statistical comparisons between the groups.

FIG. 3A. Duplex IHC for collagen type IV and TGF beta-1. Column A. In the unwounded sham surgery cornea treated with topical losartan for one month, collagen type IV was detected in Descemet's membrane (arrow), but little, if any, was detected in the stroma or stromal cells, that were primarily keratocytes. TGF beta-1 was produced in corneal endothelial cells (arrowheads) but only in a few scattered stromal cells. TGF beta-1 penetration into the stroma from the corneal endothelial cells or the aqueous humor is inhibited by the intact Descemet's membrane and its components that include collagen type IV and perlecan. The results were identical in the unwounded sham surgery group that was not treated (not shown) or the unwounded sham surgery group treated with oral vehicle alone (not shown). Column B. At one month after Descemetorhexis and one month of treatment with topical vehicle and oral vehicle, collagen type IV was present at high levels in the posterior stroma (delineated by the bracket) populated primarily with myofibroblasts and corneal fibroblasts, but not in the more anterior stroma populated primarily by keratocytes. TGF beta-1 was detected at high levels throughout the fibrotic posterior stroma in both stromal cells and stromal tissue. Column C. At one month after Descemetorhexis and one month of treatment with topical losartan alone, collagen type IV was markedly decreased in the posterior stroma, except adjacent to the posterior corneal surface (arrows) devoid of Descemet's membrane and endothelium. TGF beta-1 was present in large amounts at the posterior stromal surface and throughout the posterior stroma. Column D. At one month after Descemetorhexis and one month of treatment with oral losartan alone, collagen type IV was present at high levels in the posterior stroma populated primarily with myofibroblasts and corneal fibroblasts, but not in the more anterior stroma populated primarily by keratocytes. TGF beta-1 was detected in large amounts at the posterior stromal surface and in some stromal cells. Column E. At one month after Descemetorhexis and one month of treatment with both topical losartan and oral losartan, collagen type IV was markedly decreased in the posterior stroma. TGF beta-1 was detected at high levels at the posterior stromal surface and in some stromal cells. Blue is DAPI in all composite images. Yellow dashed rectangles show examples of areas quantitated for collagen type IV with ImageJ, with the mean of three non-overlapping rectangles taken as the value for that cornea.

FIG. 3B. Graphic results of the individual collagen type IV staining intensity in the posterior stroma analyzed with ImageJ in 0.75 W×0.5H rectangles tangent to the posterior corneal surface for each cornea for each group. Individual measurements and the mean+/−standard error for each group are shown. The double diagonal line at the top of the topical plus oral vehicle column indicates that the highest value in this group was actually beyond the units of the Y-axis (the value for this cornea was mean 6.586×10³ intensity). See Table 5 for Kruskal-Wallis test followed by post-hoc Dunn's-Bonferroni test statistical comparisons between the groups.

FIG. 4A shows the chemical structure of Losartan.

FIG. 4B shows the chemical structure of Losartan Metabolite EXP3174.

FIG. 4C shows the chemical structure of Telmisartan.

FIG. 4D shows the chemical structure of Valsartan.

FIG. 4E shows the chemical structure of Olmesartan.

FIG. 4F shows the chemical structure of candesartan.

FIG. 5A. Standardized slit lamp photos of rabbit corneas at one month after a one-minute exposure to 1N NaOH on a 5 mm diameter filter paper and continuous treatment for one month with topical vehicle (VEH), 0.2 mg/ml losartan, 1% prednisolone acetate, or 0.2 mg/ml losartan+1% prednisolone acetate six times per day. Note that opacity in each cornea is made up of central more dense (*) and peripheral less dense (**) zones. Arrows indicate central corneal neovascularization. Dotted circles show examples of ImageJ analysis of total opacity for individual corneas that include the combined more dense central and less dense peripheral zones. Mag15×.

FIG. 5B. Graph of total opacity area measured with ImageJ in individual corneas. Mean±standard error of the mean is shown for each group. * indicates the opacity was significantly different from the vehicle BSS control group. Table 8 shows Kruskal-Wallace followed by post-hoc Dunn's-Bonferroni test p-values for comparisons between the groups.

FIG. 5C. Graph of total opacity in pixels intensity measured with ImageJ in individual corneas. Mean±standard error of the mean is shown for each group. * indicates the opacity was significantly different from the vehicle BSS control group. Table 9 shows Kruskal-Wallace followed by post-hoc Dunn's-Bonferroni test p-values for comparisons between the groups.

FIG. 6A. Duplex immunohistochemistry for keratocyte-specific marker keratocan (green) and myofibroblast-specific marker α-SMA (red) in uninjured control corneas and after alkali burn injury and one month of topical treatment. Fragile peripheral epithelium and persistent epithelial defects were noted in all alkali burned corneas. In corneas treated with losartan alone or combination losartan+prednisolone acetate, α-SMA-positive myofibroblasts tended to be localized to the posterior cornea and the anterior cornea was repopulated by keratocan-positive keratocytes. Two examples of corneas treated with prednisolone acetate alone are shown to demonstrate the variability noted in this group. In #1, α-SMA-positive myofibroblasts populated the entire thickness of this cornea, with a persistent epithelial defect. In #2, α-SMA-positive myofibroblasts were present only in the posterior stroma despite the presence of a persistent epithelial defect. All alkali burned corneas were devoid of corneal endothelium over an 8 to 10 mm diameter area of the posterior cornea. Arrows indicate areas with posterior α-SMA-positive myofibroblasts. LOS is topical losartan. Pred acetate is 1% prednisolone acetate. BSS Veh is balanced salt solution vehicle. e is epithelium. S is stroma.

FIG. 6B. A graph of total α-SMA-positive stromal area determined in central sections of each cornea in each group using ImageJ. * indicates the mean was significantly different from the BSS vehicle control group. #indicates the mean was significantly different from the prednisolone acetate alone group. Table 10 shows Kruskal-Wallace followed by post-hoc Dunn's-Bonferroni test p-values for statistical comparisons between the groups.

FIG. 6C. A graph of total α-SMA-positive intensity per corneal section in pixels determined in central sections of each cornea in each group using ImageJ. * indicates the mean was significantly different from the BSS vehicle control group. #indicates the mean was significantly different from the prednisolone acetate alone group. Note the lower mean and standard error of the mean in the combined losartan+prednisolone acetate group and the higher mean and standard error of the mean in the prednisolone acetate only group. Table 11 shows Kruskal-Wallace followed by post-hoc Dunn's-Bonferroni test p-values for statistical comparisons between the groups.

FIG. 7A. Duplex immunohistochemistry for TGF beta-1 and collagen type IV in both the anterior stroma and posterior stroma. Representative IHCs for each group are shown. Arrows indicate Descemet's membrane or remnants of Descemet's membrane in each cornea. Representative ImageJ quantitation rectangles (100×50 units) for both the anterior stroma (long side of rectangle at anterior stromal surface) and the posterior stroma (long side of rectangle at posterior stromal surface just anterior to Descemet's membrane or its remnants). No differences in TGF beta-1 were noted between the groups.

FIG. 7B. ImageJ quantitation of collagen type IV IHC intensity units in the anterior stroma in the groups. * and ** indicates the mean was significantly different from the vehicle group. Table 12 shows Kruskal-Wallace followed by post-hoc Dunn's-Bonferroni test p-values for statistical comparisons between the groups.

FIG. 7C. ImageJ quantitation of collagen type IV IHC intensity units in the posterior stroma in the groups. * indicates the mean was significantly different from the vehicle group. Table 12 shows Kruskal-Wallace followed by post-hoc Dunn's-Bonferroni test p-values for statistical comparisons between the groups.

FIG. 8. Corneal angiography images at initial intravenous fluorescein filling in corneal neovascularization (CNV). Yellow lines delineate the approximate area of each cornea free of corneal neovascularization. In some eyes, the nictitating membrane or eyelid covered a portion of the peripheral cornea and the peripheral area free of CNV was approximated from direct examination at the slit lamp, as indicated. Mag. 20×.

FIG. 9. Immunohistochemistry for α-SMA in representative central sections from each cornea that were analyzed with ImageJ to determine total α-SMA-positive stromal area and total α-SMA intensity in pixels. Arrows indicate α-SMA staining in localized areas that were only in the most posterior stroma in combined losartan+prednisolone acetate treated corneas.

FIG. 10A. Slit lamp opacity after −9D PRK and topical treatment for one month with vehicle or losartan. Standardized slit lamp photos of each cornea. Note that the light reflexes are all located in similar positions. The yellow dotted circles show the 3.5 mm diameter central area within the excimer laser ablated zone of each corneal image analyzed with ImageJ to determine mean total pixels of opacity.

FIG. 10B. Graph of mean central opacity pixels within the corneas analyzed with ImageJ. Note the higher mean and higher variability (higher SEM) in the vehicle-treated corneas compared to the losartan-treated corneas.

FIG. 11A. Duplex immunohistochemistry for myofibroblast marker α-SMA and keratocyte marker keratocan in the central 0.5 mm of each cornea (labeling the same as FIG. 10A-B). For each cornea, a composite with IHC for α-SMA and keratocan, as well as DAPI staining of all cell nuclei, is shown, along with a corresponding panel showing α-SMA alone that was analyzed for total pixels of α-SMA staining with ImageJ. Yellow dashed boxes show the area of analysis with ImageJ that incorporated all α-SMA staining in the central 0.5 mm of each cornea. * denotes examples of artifactual dislocations of the epithelium from the stroma that occurred in most corneas during sectioning—which was likely related to defective regeneration of the EBM that is present in nearly all rabbit corneas that have −9D PRK at one month after surgery.6,7 Blue is DAPI staining of cell nuclei in all panels. Cells in the stroma in many panels in both groups that had DAPI-positive nuclei, but which were SMA-negative and keratocan-negative, were likely primarily corneal fibroblasts, although some immune cells can also persist at one month after PRK.

FIG. 11B. A graph of total α-SMA pixels determined with the ImageJ analyses of the rectangles shown in A. Note the higher mean in the vehicle-treated group compared to the losartan-treated group and also the greater variation (higher SEM) in the vehicle-treated group.

FIG. 12A. Representative duplex immunohistochemistry for collagen type IV (green) and TGF beta-1 (magenta) in the central cornea. In the unwounded cornea, collagen type IV (COL IV) localized primarily to the epithelial basement membrane (arrows) and Descemet's membrane (arrowheads), with little detected in the stroma. In the vehicle-treated group at one month after −9D PRK, COL IV was present in a broad band posterior to the epithelial basement membrane (indicated by arrows), as well as in the uninjured Descemet's membrane. In the losartan-treated group one month after −9D PRK, COL IV was localized to the epithelial basement membrane (arrows), and only a relatively small quantity was detected in the underlying stroma. Descemet's membrane (not shown for this losartan-treated cornea) also had large amounts of COL IV. In the COL IV only panel for each −9D PRK cornea, the dotted rectangle is the 900-pixel by 235-pixel analysis rectangle in ImageJ that enclosed all stromal collagen type IV in each cornea in both treatment groups. e is epithelium. Blue is DAPI in all panels.

FIG. 12B. A graph of total COL IV pixels determined with the ImageJ analyses of the rectangles (as is shown in A). Note the marked downregulation of the total COL IV staining pixels in the losartan-treated group compared to the vehicle-treated group (p=0.004).

### DEFINITIONS

As used herein, the terms “host,” “subject” and “patient” refer to any animal, including but not limited to, human and non-human animals (e.g., dogs, cats, cows, horses, sheep, poultry, etc.) that is treated, studied, analyzed, tested, or diagnosed.

### DETAILED DESCRIPTION

The present invention relates to compositions, systems, and methods for treating a subject with an eye condition (e.g., topically) using a composition comprising an ACE-2 receptor antagonist (also known as Angiotensin II Receptor Blocker or “ARB”) (e.g., losartan, telmisartan, valsartan, olmesartan, candesartan, irbesartan, eprosartan, azilsartan, or losartan metabolite EXP3174). In certain embodiments, the eye condition is selected from: i) a corneal scarring fibrosis, ii) a transforming growth factor beta-induced (TGFBI) corneal dystrophy, iii) a conjunctival fibrotic disease, iv) an intraocular fibrotic disease, and v) a conjunctival bleb scarring and/or shunt encapsulation (e.g., following glaucoma surgery).

In work conducted during development of embodiments of the present disclosure, it was shown that topical losartan could be effective in limiting myofibroblast development and fibrosis triggered by more anterior to mid-corneal injuries such as trauma, chemical burns, microbial infections and surgeries, such as photorefractive keratectomy or phototherapeutic keratectomy or more posterior corneal injuries such as endothelial trauma during cataract or other surgery, Descemetorhexis, or endotheliitis. Trauma, chemical burns, microbial infections, surgeries, or diseases could involve any layer of the cornea. In other work conducted during development of embodiments of the present disclosure, it was a shown that the combination of an ACE-2 receptor antagonist and a corticosteroid was effective in treating eye trauma.

In certain embodiments, the ACE-2 receptor antagonist and/or corticosteroid are present in one or more compositions that comprise water (e.g., about 90% water) and an oily excipient (e.g., to benefit the ocular surface by restoring the lipid layer of the tear film and protecting the aqueous layer from drying out). In some embodiments, the compositions are in the form of an oil-in-water emulsion (e.g., like Restasis®, Lacrinmune® and Ikervis®, but containing an ACE-2 receptor inhibitor and/or corticosteroid instead of CsA) or micelle based solution (e.g., like Papilock Mini®, Modusik-A Ofteno® and Taejoon [TJ]Cyporin®, but containing an ACE-2 receptor inhibitor and/or corticosteroid instead of CsA). In particular embodiments, the compositions contain one or more of the following: i) a solubilizing agent/enhancer (e.g., castor oil, medium-chain triglycerides, polyoxly-40 stearate ethanol, polysorbate 80 ethanol, or corn oil), ii) a surfactant (e.g., polysorbate 80, tyloxapol, poloxamer 188, or cetalkonium chloride), iii) a preservative (e.g., boric acid, or sorbic acid), iv) a stabilizer (e.g., carbomer copolymer type A, or sodium EDTA), v) a viscosity regulator (e.g., hypromellose, or sodium hyaluronate), vi) pH regulator (e.g., NaOH, NaH₂PO₄, or sodium bisulfite), vii) an osmotic agent (e.g., glycerol or NaCl), and/or viii) a diluent (e.g., water, petrolatum, lanolin, or alcohol).

In certain embodiments, the improvement of a corneal injury (compared to if left untreated) or existing corneal scar that is treated is measured by the Fantes slit-lamp corneal haze score, as described in Fantes et al., Arch. Opthalmol., 1990, 108(5):665-675 (herein incorporated by reference) and shown in Table 6 below:

TABLE 6

| | |
|---|---|
| 0 | Clear with no opacity seen by any method of microscopic |
| | slit-lamp examination |
| 0.5* | Trace or faint haze seen only by indirect, broad tangential |
| | illumination |
| 1 | Haze of minimal density seen with difficulty with direct or |
| | diffuse examination |
| 2 | Mild haze easily visible with direct focal slit lamp illumination |
| 3 | Moderate opacity that partially obscured details of iris |
| 4 | Severe opacity chat completely obscured the details of |
| | intraocular structures |

Notes:
*G0.5 considered clear. Data from Fantes et al.¹⁵
Corneal haze grading scale

While the present disclosures is not limited to any particular mechanism, it is believed that oral losartan described in the Example below, was not effective in decreasing corneal scarring fibrosis after DMR because it didn't reach sufficient concentration in the corneal stroma compared to the concentrations achieved with topical delivery. That is not surprising since oral losartan has been used by millions of patients for hypertension or other diseases since its approval by the FDA in 1995, and no beneficial effects on corneal scarring fibrosis have been reported.

Damage to the corneal epithelium, such as by abrasion or other trauma, is quickly repaired (usually within 24-48 hours) by growth of rapidly dividing epithelial cells. However, this rapid proliferation of corneal epithelial cells is frequently accompanied by the development of scar tissue. The presence of scar tissue in the cornea results in ‘corneal haze’—an opacification of the cornea in which vision is dramatically reduced due to the inability of light to pass through the cornea. Treatment of corneal opacification varies with the extent of scar tissue formation. In cases where the scarring remains light and affects only the surface of the cornea, surgery or laser removal is used as treatment. In situations where the scar tissue extends deeper into the cornea removal of the entire tissue and transplantation of a new cornea is often used. Prevention of scarring in this tissue after injury is thus a critical step in the preservation of vision.

A number of corneal injuries are known to typically produce scarring of the cornea. These fall into three broad categories: trauma, infection, and disease conditions, all of which are contemplated to be treated by the drug agents herein. Natural traumas (such as abrasion or chemical burns), as well as trauma associated with medical correction of vision (such as photoablation, or contact lens-induced injury) cause disruption of the normal corneal epithelium, resulting in rapid growth of these cells and often formation of scar tissue. Damage to the cornea resulting from surgery, such as transplantation, also commonly leads to scarring of this tissue.

Infections of the eye by bacteria, viruses, fungi, acanthamoeba and other organisms can also lead to scarring. For example, ocular infection by herpes simplex virus type I, Pneumococcus, Staphylococcus, Escherichia coli, Proteus, Klebsiella and Pseudomonas strains are known to cause ulcer formation on the surface of the cornea. Such ulcers not only destroy the surrounding epithelial layer, but also penetrate and damage the corneal stroma, further aided by acute inflammatory cells and collagenase released by the injured epithelial cells themselves. Such deep and extensive damage to the cornea and surrounding tissues results in extensive scarring. Other, non-ulcerative pathogens are also known to lead to scarring of the cornea. One such organism is herpes zoster virus (shingles); infection by this organism frequently result in scarring.

A number of disease conditions not immediately caused by a pathogen or trauma have also been implicated in corneal opacification due to scarring. Two such conditions are cicatricial pemphigoid and Stevens-Johnson syndrome (SJS). Cicatricial pemphigoid is an autoimmune blistering disease affecting oral mucosa and the conjunctiva of the eye, in which inflammation of the corneal epithelium leads to scarring. SJS is a severe form of erythema multiforme, an immune complex-mediated hypersensitivity reaction. The ocular manifestation of this disease is ulceration of the epithelium, followed by severe scarring.

A majority of patients develop various degrees of corneal haze following excimer photorefractive keratectomy (PRK). Corneal haze typically peaks at two to four months and has been noted to increase with the degree of myopia corrected. Such haze can lead to the loss of one or more lines of best corrected visual acuity after PRK. Corneal stromal remodeling influences the degree of corneal haze after PRK and corneal haze is believed to be responsible for a reduction in the best possible corrected visual acuity, regression for refractive correction and poor predictability for the attempted correction. The formation of the corneal haze after PRK is a result of laser corneal ablation and stromal wound healing. Despite significant advances made in understanding PRK technology (e.g., laser-tissue interaction, optical profiling of the laser beam, multi-zone multi-pass approaches and edge-smoothing techniques), characterization of biological aspects associated with PRK, such as wound healing, remains a significant limitation associated with PRK technology.

In some embodiments, the eye conditions listed in Table 15 below may be treated with an ACE-2 receptor antagonist (e.g., Losartan), whether in conjunction with other treatments or alone.

TABLE 15

| Disease or Disorder | | |
|---|---|---|
| Category | Specific disease or disorder | e.g., Adjuvant to |
| Corneal scarring | | |
| fibrosis | | |
| | Descemetorhexis without graft | |
| | DSAEK or DMEK graft displacement | |
| | Alkali or acid burns | Topical corticosteroids |
| | Sulfur mustard or other chemical | Topical corticosteroids |
| | weapons burns | |
| | PRK late haze | |
| | PRK breakthrough late haze after | |
| | MMC treatment | |
| | LASIK complications scars | |
| | Persistent epithelial defects | Oxervate (NGF) |
| | Recurrent Herpes simplex | Oral acyclovir, |
| | virus (HSV) keratitis | valacyclovir or ganciclovir |
| | HSV endotheliitis with scarring | Oral acyclovir or |
| | | valacyclovir |
| | Herpes zoster ophthalmicus | Oral acyclovir or |
| | | valacyclovir |
| | Bacterial keratitis | antibiotics |
| | Fungal keratitis | antifungals |
| | Acanthamoeba keratitis | Anti-acanthamoeba |
| | | agents |
| | Corneal lacerations with | |
| | excessive fibrosis | |
| TGFBI corneal | | |
| dystrophies | | |
| | Reis-Bücklers corneal dystrophy | |
| | Thiel-Behnke corneal dystrophy | |
| | Lattice corneal dystrophy type 1 | |
| | Granular corneal dystrophy | |
| | type 1 and type 2 | |
| Conjunctival fibrotic diseases | Trachoma | Azithromycin |
| | Stevens-Johnson syndrome | Corticosteroids |
| | Ocular cicatricial pemphigoid | Dapsone |
| | Ocular graft-vs-host disease | Corticosteroids |
| Intraocular fibrotic | Proliferative vitreoretinopathy | Corticosteroids, |
| diseases | | anti-VEGF compounds |
| Conjunctival bleb scarring | | Corticosteroids |
| and shunt encapsulation | | |
| (e.g., following | | |
| glaucoma surgery) | | |

In certain embodiments, the compositions herein containing an ACE-2 receptor antagonist (e.g., Losartan) are used to regulate posterior corneal fibrosis that can develop after endothelial replacement surgeries such as, for example, DSAEK and DMEK,^{31,32} especially if the transplanted tissue becomes partially dislocated from its intended bed such that an area of the posterior stroma is not covered with Descemet's membrane and endothelium.^{19,22} In corneal disorders associated with severe inflammation, such as alkali bums, the compositions herein containing an ACE-2 receptor antagonist may be very effective treatment since the corticosteroid also triggers apoptosis of fibrocyte myofibroblast precursor cells²⁰ and severe inflammation damages corneal stromal fibrils and matrix that also underlies corneal opacity.²² Some other exemplary corneal disorders where the compositions herein (containing an ACE-2 receptor antagonist) may be effective prophylactically is to inhibit the development of stromal fibrosis and therapeutically to treat fibrosis once it has developed include viral, bacterial, fungal, and acanthamoeba keratitis, corneal trauma, chemical burns, and other corneal surgery-induced fibrosis. The compositions herein could also be used to inhibit fibrosis in other areas of the anterior segment of the eye where myofibroblasts have a role in the pathophysiology, such as conjunctival scarring disorders like trachoma,³³ and the fibrosis of conjunctival blebs after glaucoma filtering surgeries.³⁴

### EXAMPLES

### Example 1

Topical Losartan Inhibits Corneal Scarring Fibrosis after Injury

This Example describes examining the effect of topical and/or oral TGF-beta blocker losartan on corneal stromal fibrosis that developed in rabbit corneas after Descemetorhexis removal of central Descemet's membrane and corneal endothelium. Twenty-eight New Zealand white rabbits were included and either had 8 mm central Descemetorhexis or shame control surgery without Descemetorhexis in one eye. Groups of 4 eyes without Descemetorhexis were treated for one month with no medications, topical losartan or oral losartan. Groups of 4 eyes with Descemetorhexis were treated with topical and oral vehicle, topical losartan, oral losartan, or both topical losartan and oral losartan for one month. Standardized slit lamp photos were obtained with central opacity intensity measured with ImageJ. The posterior fibrotic zone of corneas was measured on immunohistochemistry for alpha-smooth muscle actin (SMA) and keratocan using QuPath analysis. Collagen type IV expression in the posterior cornea was quantitated with ImageJ and duplex immunohistochemistry for collagen type IV and TGF beta-1. After Descemetorhexis, topical, but not oral, losartan decreased the intensity of central stromal opacity and reduced peripheral corneal scarring compared to corneas that had Descemetorhexis and treatment with vehicles alone. Topical losartan also reduced corneal neovascularization that developed after Descemetorhexis. Topical losartan decreased posterior stromal cellular collagen type IV production compared to vehicle treatment at one month after Descemetorhexis.

Myofibroblast development and scarring stromal fibrosis is mediated by transforming growth factor (TGF) beta, and likely other growth factors such as platelet-derived growth factor (PDGF), after both anterior and posterior injuries to the cornea (de Oliveira et al., 2021; Wilson, 2019). A recently characterized model for posterior corneal fibrosis is Descemetorhexis removal of a portion of the Descemet's membrane-endothelial complex in rabbits (Sampaio et al., 2021). Aqueous humor is likely the dominant source of TGF beta-1 and TGF beta-2 that subsequently drive the development of myofibroblasts from corneal fibroblasts and fibrocytes in the absence of the growth factor modulatory function of Descemet's basement membrane (Medeiros et al., 2020; Sampaio et al., 2021).

Losartan is an oral medication that is used to treat high blood pressure, diabetic kidney disease, heart failure, and left ventricular enlargement (Simpson and McClellan, 2000). Losartan is an angiotensin II receptor antagonist (Michel et al., 2013), but has also been shown to be an inhibitor of TGF beta (Lim et al., 2001; Lavoie et al., 2005; Cohn et al., 2007; Wylie-Sears et al., 2014; Geirsson et al., 2012; Park et al., 2012). The purpose of this study was to determine if losartan inhibited scarring fibrosis after corneal injury using a rabbit Descemetorhexis model previously shown to trigger posterior corneal myofibroblast development and fibrosis. This study shows that topical, but not oral, losartan significantly decreases stromal opacity, stromal fibrosis, and stromal collagen type IV production after Descemetorhexis injury in rabbits.

### METHODS

### Animals

All procedures involving animals were approved by the Institutional Animal Care and Use Committee (IACUC) at the Cleveland Clinic Foundation (Cleveland, OH, USA) and the Animal Care and Use Review Office of the Department of the Army (Fort Detrick, MD). All animals were treated in accordance with the tenets of the ARVO Statement for the Use of Animals in Ophthalmic and Vision Research. Twenty-eight 10- to 15-week-old female New Zealand White rabbits weighing 2.5 to 3.0 kg each were included in this study—four in each group.

### Excision of the Central Descemet's Membrane (DM)-Endothelial Complex (Descemetorhexis)

The rabbits had general anaesthesia with 30 mg/kg ketamine hydrochloride and 5 mg/kg xylazine by IM injection. In addition, topical proparacaine hydrochloride 1% (Alcon, Ft Worth, TX, USA) was applied to each eye prior to the surgery. One cornea selected at random in each group either had sham surgery without Descemetorhexis or an 8 mm diameter central Descemet's membrane-endothelial excision. The time point of one month after surgery was selected for the study because myofibroblasts and fibrosis in the posterior cornea was maximal at this time point in a prior time course Descemetorhexis study (Sampaio et al., 2021). Descemetorhexis was performed as described in the prior study (Sampaio et al., 2021). Briefly, a wire lid speculum was placed into one eye of each rabbit and an 8 mm diameter gentian violet circle was marked on the epithelial surface for reference. A limbal entry incision was created with a 1.6 mm blade (Bausch and Lomb, Rochester, NY, USA) and 0.3 ml Healon OVD (Abbott Medical Optics Inc. Santa Ana, CA, USA) was injected into the anterior chamber. A reversed Sinskey hook (Katena, Denville, New Jersey, USA) was placed into the anterior chamber and an 8 mm diameter disc of Descemet's membrane and endothelium was excised underlying the 8 mm area previously marked on the anterior corneal surface, without a subsequent endothelial corneal transplant. No removal of Descemet's membrane or endothelium was performed in unwounded sham surgery eyes. Remaining Healon OVD was removed with a Simcoe irrigation and aspiration cannula (Bausch & Lomb, Storz, Rochester, NY, USA) and the anterior chamber was filled with balanced salt solution (BSS). A single 10-0 nylon suture was placed at the incision site to prevent leakage from the anterior chamber. One drop of ciprofloxacin (Alcon, Ft. Worth, TX, USA) was applied to the corneas immediately after surgery and four times a day until the epithelium entry wound healed at approximately 2 days.

Beginning immediately after surgery, depending on the group (Table 1), Descemetorhexis and unwounded control eyes were treated with 30 μl 0.1 mg/ml losartan (Merck and Co, Kenilworth, NJ) in normal saline pH 7.0 or 30 μl of vehicle normal saline pH 7.0 six times per day (every two hours from 8 am to 6 pm).

TABLE 1
Study groups

| | | Number of |
|---|---|---|
| Group | Treatment | eyes |
| 1 | Unwounded no medications | 4 |
| 2 | Unwounded topical 0.1 mg/ml losartan 6X per day | 4 |
| 3 | Unwounded oral 5 mg/kg losartan 3X per day | 4 |
| 4 | DMR topical vehicle 6X per day and oral | 4 |
| | vehicle 3X per day | |
| 5 | DMR topical 0.1 mg/ml losartan 6X per day | 4 |
| 6 | DMR oral 5 mg/kg losartan 3X per day | 4 |
| 7 | DMR topical 0.1 mg/ml losartan 6X per | 4 |
| | day + oral 5 mg/kg losartan 3X per day | |

Animals that received oral losartan were administered 5 mg/kg in oral solution or oral vehicle solution alone three times per day at 8 am, 1 μm and 6 pm using an oral applicator with a syringe. The oral solution was prepared with 5 ml distilled water+50 ml ora-plus (suspending vehicle—Perrigo, Dublin, Ireland)+45 ml Ora-sweet (flavored syrup vehicle—Perrigo).

### Standardized Slit Lamp Photographs and ImageJ Analysis of Scar Intensity

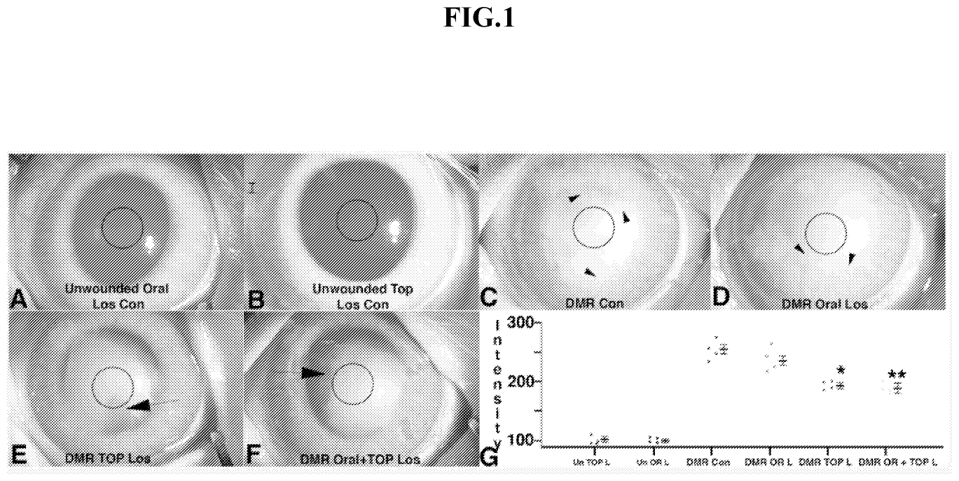

After one month of treatment, study eyes were dilated with two drops of 1% tropicamide (Akom Co., Lake Forest, IL) for thirty minutes. After general anaesthesia, the study eye in each rabbit had standardized slit lamp photographs at 20× magnification with a Haag Streit (Mason, OH, USA) BX900 slit lamp photography system using identical lighting position and intensity. For each study eye, the intensity of a 2.5 mm diameter central circle (not including the light reflex, examples shown in FIG. 1) was determined using ImageJ 1.53a analysis software. Statistical comparisons of the intensity of opacity was performed using the Kruskal-Wallis test followed by post-hoc Dunn's-Bonferroni test and p<0.05 was considered statistically significant

### Corneal Fixation, Immunohistochemistry, and Fibrotic Area Analysis

At one month after surgery and treatment, an intravenous injection of 100 mg/kg Beuthanasia (Shering-Plough, Kenilworth, NJ) was given for euthanasia with the animals under general anaesthesia. Sharp Westcott scissors (Fairfield, CT, USA) and 0.12 forceps (Storz, St. Louis, MO) were used to remove the comeo-scleral rims of the sham unwounded and DMR eyes without touching the cornea. Each cornea was set in the center of a 24×24×5 mm mold (Fisher Scientific, Pittsburgh, PA, USA) and the mold was filled in liquid OCT compound (Sakura Finetek, Torrance, CA, USA). The molds and cornea-scleral rims were quick frozen on dry ice and stored at −80° C. until sections were cut.

Each OCT block was bisected in the exact center of the cornea, and 8 μm thick transverse sections were cut from the central cornea within the previous DMR injury or unwounded sham surgery corneas with a cryostat (HM 505M; Micron GmbH, Walldorf, Germany) and three sections per slide were placed on 25 mm×75 mm×1 mm Superfrost Plus microscope slides (Fisher Scientific, Pittsburgh, PA, USA). Slides were maintained at −20° C. until immunohistochemistry.

Duplex immunohistochemistry (IHC) was performed after masking of slides by a researcher not involved in the analysis using previously described methods (de Oliveira et al., 2021) using primary antibodies (Table 2) confirmed by western blotting and IHC to recognize rabbit antigens or isotypic control antibodies (ThermoFisher Scientific, Waltham, MA), and secondary fluorescent tagged antibodies (Table 3).

TABLE 2
Primary Antibodies

| | | Species | Ab iso- | Catalog | |
|---|---|---|---|---|---|
| Antigen | Source | source | type | number | Dilution |
| TGFβ1 | Genetex | Mouse | IgG₁ | GTX21279 | 1:100 |
| Keratocan | Winston | Goat | IgG | — | 1:200 |
| | Kao, PhD | | | | |
| Vimentin | Abcam | Chicken | IgY | Ab24525 | 1:20000 |
| SMA | Dako | Mouse | IgG₂ₐ | M0851 | 1:400 |
| Collagen IV | Millipore | Goat | IgG1 | AB769 | 1:1200 |

TABLE 3
Secondary Antibodies

| | Source | Species | | Catalog | |
|---|---|---|---|---|---|
| Antibody | company | source | Isotype | number | Dilution |
| Alexa Fluor | Thermo Fisher | donkey | IgG | A21202 | 1:200 |
| 488 | Scientific | | | | |
| anti-mouse | | | | | |
| Alexa Fluor | Thermo Fisher | donkey | IgG | A11055 | 1:200 |
| 488 | Scientific | | | | |
| anti-goat | | | | | |
| Alexa Fluor | Thermo Fisher | donkey | IgG | A10037 | 1:200 |
| 568 | Scientific | | | | |
| anti-mouse | | | | | |
| Alexa Fluor | Thermo Fisher | donkey | IgG | A11057 | 1:200 |
| 568 | Scientific | | | | |
| anti-goat | | | | | |

The AB769 (Millipore, Temecula CA) collagen type IV antibody used in this study was generated against purified human and bovine collagen type IV, affinity purified with human and bovine collagen type IV crosslinked to agarose and cross-absorbed by the manufacturer with human and bovine collagens type I, II, III, V and VI to eliminate cross-reactivity. This antibody was shown previously to bind rabbit collagen IV in IHC (Sampaio et al., in press). The keratocan antibody was raised against peptide H2N-LRLDGNEIKPPIPIDLVAC-OH (SEQ ID NO:1). The antibody to TGF beta-1 (GeneTex, Irvine, CA) binds rabbit TGF beta-1 in IHC and shows no reactivity to TGF beta-2 or TGF beta-3 (de Oliveria et al., 2021).

The area of the SMA-positive posterior fibrotic stroma was quantitated with the original groups masked to the analyzer by standardized photography with a lOx objective on a Leica DM6B upright microscope equipped with an automated stage and Leica 7000 T camera using the LAS X software (Leica Microsystems, GmbH, Wetzlar, Germany). The area of the SMA-positive posterior stromal fibrosis was measured by manually outlining the area on the cornea sections with calibrated QuPath v0.2.3 software using previously described methods (Bankhead et al., 2017). Statistical analysis for the area of SMA-positive fibrosis was performed using the Kruskal-Wallis test followed by post-hoc Dunn's-Bonferroni test and p<0.05 was considered statistically significant.

Collagen type IV intensity in immunohistochemistry images was quantitated in 0.75 W×0.5H rectangles of posterior stroma tangent to the posterior corneal surface using ImageJ on 7.62 cm wide×5.69 cm height and 300 DPI images generated using standardized microscopic illumination settings applied to all sections. The quantitation rectangles were positioned such that the posterior side of the rectangle was tangent to the posterior corneal surface just anterior to Descemet's membrane, if one was present, in the analysed specimens. The mean collagen type IV intensity of three non-overlapping rectangles within the Descemetorhexis or corresponding area of sham unwounded corneas was used as the value for that cornea. p-values for the statistical comparisons between all the groups were performed with the Kruskal-Wallis test followed by post-hoc Dunn's-Bonferroni test.

### Results

### Slit Lamp Photographs and Opacity in the Central Cornea

Representative standardized slit lamp photos from the different groups at one month after treatment are shown in FIG. 1. Either oral (A) 5 mg/kg losartan three times per day or 0.1 mg/ml topical (B) losartan six times per day had no effect on the transparency of sham unwounded corneas. At one month after Descemetorhexis and treatment with oral vehicle solution three times per day and topical vehicle solution six times per day (C), all corneas had severe scarring fibrosis extending to the limbus with prominent neovascularization. At one month after Descemetorhexis and treatment with 5 mg/kg losartan orally three times a day for one month (D), there was no difference in scarring fibrosis and neovascularization compared to the control corneas that had Descemetorhexis and vehicle treatment. At one month after Descemetorhexis and treatment with topical 0.1 mg/ml losartan six times per day for one month (E), there was an overall decrease in scarring fibrosis, with a decrease in the intensity of the central opacity, transparency in the peripheral cornea that allowed iris details to be clearly seen, and areas of clearing in the mid-stromal opacity. At one month after Descemetorhexis and treatment with both topical 0.1 mg/ml losartan six times per day and oral 5 mg/kg losartan three times a day for one month (F), the changes in corneal opacity and neovascularization were not different from those noted with treatment with topical losartan alone.

When the intensity of opacity in a 2.5 mm circle of central cornea was quantitated with ImageJ (FIG. 1G) there was a significant decrease (P<0.05 Kruskal-Wallis test followed by post-hoc Dunn's-Bonferroni test) in the group that had Descemetorhexis with topical losartan for one month or Descemetorhexis with topical losartan and oral losartan for one month compared to the group that had Descemetorhexis with topical vehicle and oral vehicle solutions for one month or the group that had Descemetorhexis with oral losartan alone for one month. There was no significant difference in the intensity of opacity in the central cornea between the group that had Descemetorhexis with topical losartan for one month and the group that had Descemetorhexis with topical losartan and oral losartan for one month.

### Immunohistochemistry for α-Smooth Muscle Actin (SMA) Myofibroblast Marker and Keratocan Keratocyte Marker

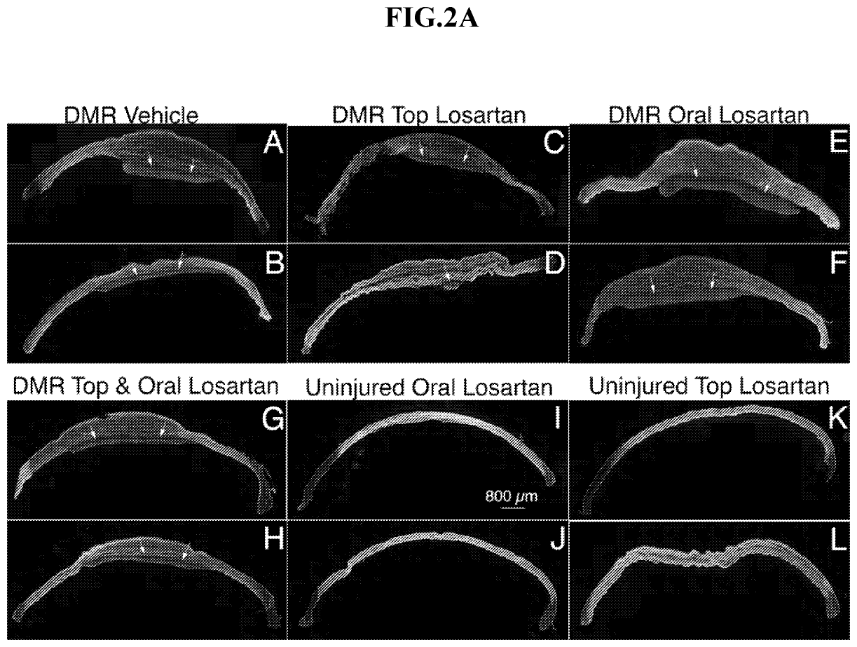

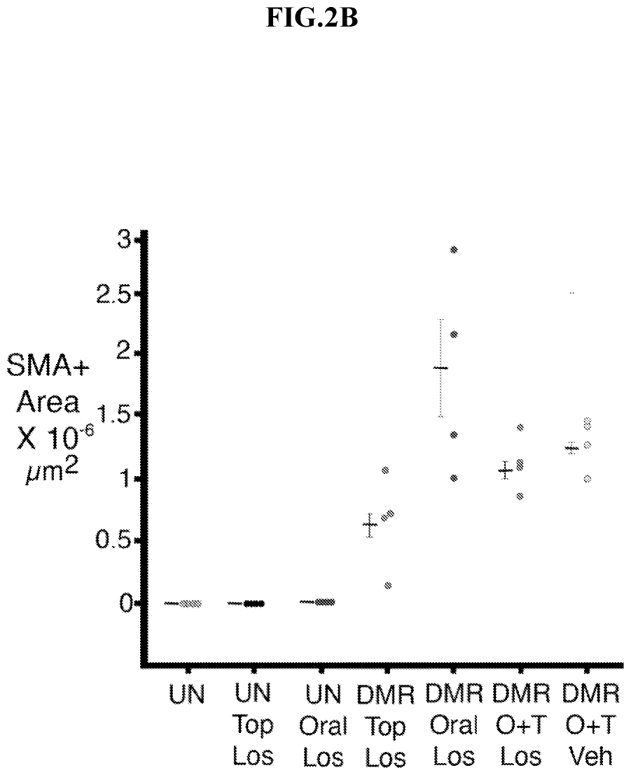

Representative results for immunohistochemistry for SMA and keratocan are shown in FIG. 2. Corneas that had Descemetorhexis and treatment with topical vehicle and oral vehicle for one month (A, B) had prominent posterior corneal fibrosis delineated by IHC for the SMA myofibroblast marker. Corneas that had Descemetorhexis and treatment with topical 0.1 mg/ml losartan six times per day for one month (C, D) all had a marked decrease in the SMA-positive fibrotic zone in the posterior cornea. The cornea shown in FIG. 2D had the smallest area of posterior fibrosis in any cornea that had Descemetorhexis in any group. Corneas that had Descemetorhexis and treatment with oral 5 mg/kg losartan three times per day for one month (E, F) had posterior fibrosis that was similar to corneas that had Descemetorhexis treated with topical and oral vehicle solutions for one month. Corneas that had Descemetorhexis and were treated with both topical losartan six times per day and oral losartan three times per day for one month (G, H), also had a decrease in the SMA-positive fibrosis in the posterior cornea but were not significantly different from the corneas that had Descemetorhexis and treatment with topical losartan alone. Uninjured corneas treated either with oral losartan three times a day (I, J) or topical losartan six times a day (K, L) did not develop SMA-positive posterior corneal fibrosis.

The graph in FIG. 2M shows the results of quantitation of the area of the posterior fibrotic zone in each cornea in each group. There was no SMA-positive fibrosis in any of the uninjured corneas, whether they received no treatment, topical losartan six times per day or oral losartan three times per day. In the groups that had Descemetorhexis, those treated with topical 0.1 mg/ml losartan six times a day had significantly lower (Table 4) mean SMA-positive posterior fibrotic area compared to the other groups that had Descemetorhexis, whether they were treated with oral losartan alone for one month, oral and topical losartan for one month or oral and topical vehicle solutions for one month. The mean area of the SMA-positive posterior fibrotic area was highest in the group treated with oral losartan alone for one month, and the variability in the area of SMA-positive posterior fibrosis was also greater in that group. However, the mean SMA-positive posterior fibrotic area in the Descemetorhexis corneas treated with oral losartan alone was not statistically significantly different from the groups that had Descemetorhexis treated with topical and oral vehicles for one month or treated with topical and oral losartan for one month. Table 4 provides the p-values for the statistical comparisons between all the groups performed with the Kruskal-Wallis test followed by post-hoc Dunn's-Bonferroni test.

TABLE 4

| | | | | | | DMR | DMR |
|---|---|---|---|---|---|---|---|
| | Unwounded | Unwounded | Unwounded | DMR | DMR | TOP and | TOP and |
| | untreated | Top Los | Oral Los | Top Los | Oral Los | Oral Los | Oral Veh |
| Unwounded | X | 1 | 1 | 0.01 | 0.01 | 0.01 | 0.01 |
| untreated | | | | | | | |
| Unwounded | | X | 1 | 0.01 | 0.01 | 0.01 | 0.01 |
| Top Los | | | | | | | |
| Unwounded | | | X | 0.01 | 0.01 | 0.01 | 0.01 |
| Oral Los | | | | | | | |
| DMR Top | | | | X | 0.04 | 0.04 | 0.04 |
| LOS | | | | | | | |
| DMR Oral | | | | | X | 0.25 | 0.39 |
| LOS | | | | | | | |
| DMR TOP | | | | | | X | 0.25 |
| and Oral Los | | | | | | | |
| DMR Top | | | | | | | X |
| and Oral Veh | | | | | | | |

### Immunohistochemistry Collagen Type IV and TGF Beta-1, and Quantitation of Posterior Stromal Collagen Type IV Staining Intensity

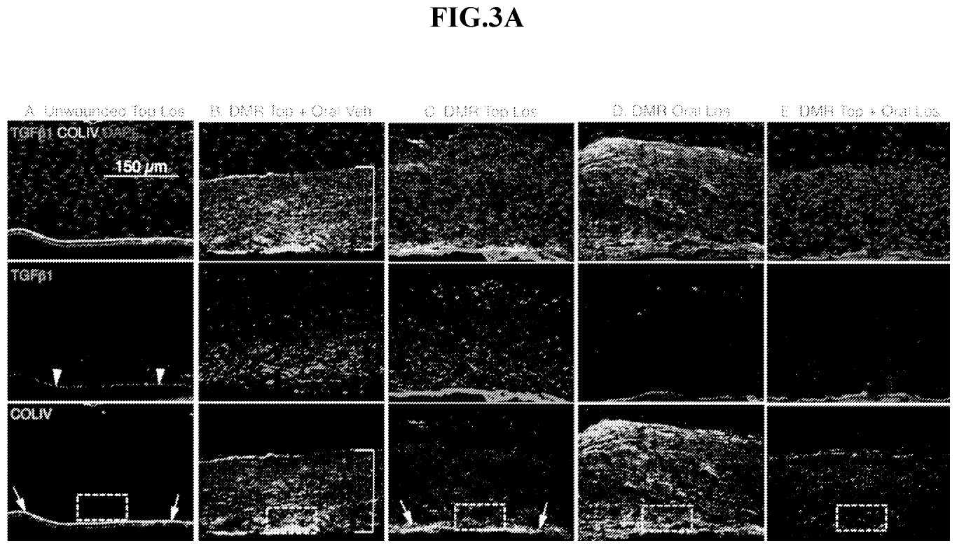

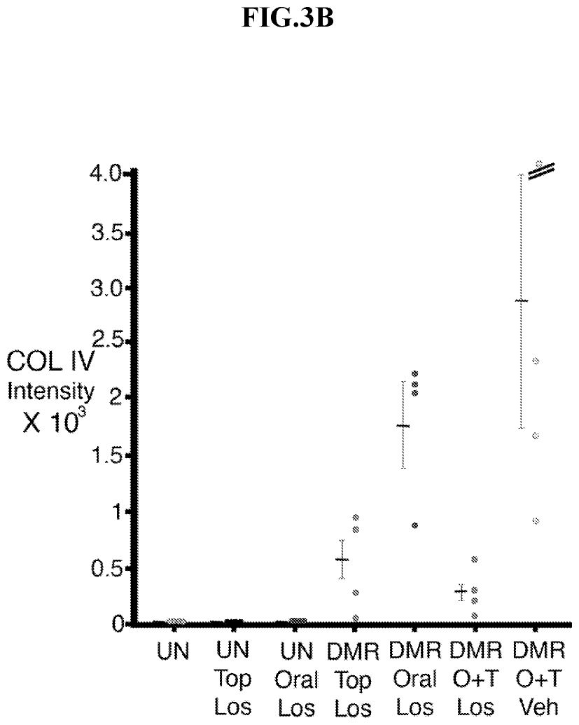

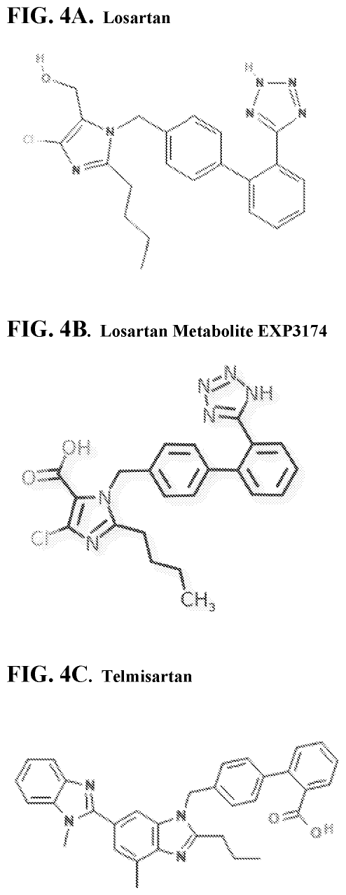

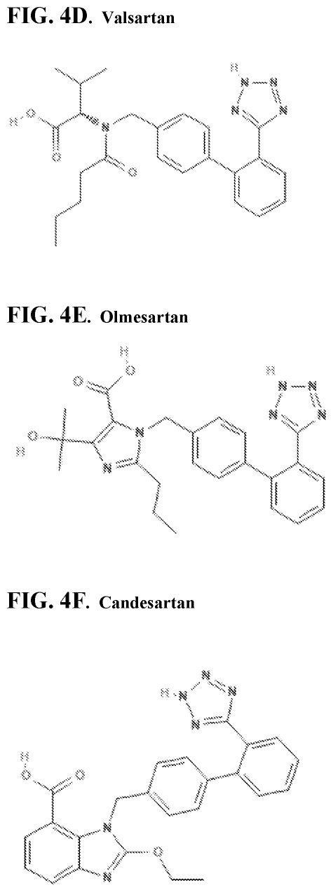

Representative results for immunohistochemistry for collagen type IV, in duplex with TGF beta-1, are shown in FIG. 3. In sham unwounded corneas that were treated with topical losartan (A) or oral losartan (not shown), or were not treated (not shown), collagen type IV localized primarily to Descemet's basement membrane and the epithelial basement membrane (not shown), with only rare stromal cells containing collagen type IV. TGF beta-1 was localized to the endothelium, and rare stromal cells, as well as to the corneal epithelium (not shown).

All Descemetorhexis corneas at one month after surgery were devoid of central Descemet's membrane and corneal endothelium. In corneas that had Descemetorhexis and treatment with both topical vehicle and oral vehicle for one month (FIG. 3B) the posterior stroma contained a thick posterior fibrotic zone filled with SMA-positive myofibroblasts and an overlying layer with SMA-negative, keratocan-negative cells that were primarily corneal fibroblasts (this can be seen in FIG. 2). These cells populate the posterior fibrotic zone shown with brackets in FIG. 3B, and this entire zone had high levels of collagen type IV. Many of the cells in the fibrotic zone in these corneas had TGF beta-1 associated with them. In corneas that had Descemetorhexis and treatment with topical losartan alone for one month (FIG. 3C), collagen type IV was primarily detected adjacent to the posterior surfaces. Many cells in the posterior stroma of these corneas had associated TGF beta-1. Some, but not all, of these corneas also had a dense layer of TGF beta-1 accumulation at the posterior corneal 5 surface, as is shown in the cornea shown in FIG. 3C. Corneas that had Descemetorhexis and were treated with oral losartan alone (FIG. 3D) were not significantly different from corneas that had Descemetorhexis and treatment with topical vehicle and oral vehicle for one month (FIG. 3B). Corneas that had Descemetorhexis and were treated with both topical losartan and oral losartan for one month (FIG. 3E) were not different as a group in collagen type IV from corneas that had Descemetorhexis and were treated with topical losartan alone. Thus, oral losartan alone had no significant effect on collagen type IV production in the posterior stroma and oral losartan treatment had no additive effect to topical losartan alone treatment.

FIG. 3F is a graph showing the collagen type IV intensity quantitated in 0.75 w×0.5h ImageJ rectangles of posterior stroma tangent to the posterior corneal surface, and not including Descemet's membrane in unwounded corneas, as shown in FIG. 3A to 3E. Topical losartan or topical losartan and oral losartan treatment significantly decreased posterior stromal collagen type IV production at one month after Descemetorhexis compared to corneas treated with topical vehicle and oral vehicle.

Table 5 provides the p-values for the statistical comparisons between all the groups in collagen type IV levels in the posterior stroma performed with the Kruskal-Wallis test followed by post-hoc Dunn's-Bonferroni test.

TABLE 5

| | | | | | | DMR | DMR |
|---|---|---|---|---|---|---|---|
| | Unwounded | Unwounded | Unwounded | DMR | DMR | TOP and | TOP and |
| | untreated | Top Los | Oral Los | Top Los | Oral Los | Oral Los | Oral Veh |
| Unwounded | X | 1 | 1 | 0.01 | 0.01 | 0.01 | 0.01 |
| untreated | | | | | | | |
| Unwounded | | X | 1 | 0.01 | 0.01 | 0.01 | 0.01 |
| Top Los | | | | | | | |
| Unwounded | | | X | 0.01 | 0.01 | 0.01 | 0.01 |
| Oral Los | | | | | | | |
| DMR Top | | | | X | 0.08 | 0.56 | 0.04 |
| LOS | | | | | | | |
| DMR Oral | | | | | X | 0.02 | 0.56 |
| LOS | | | | | | | |
| DMR TOP | | | | | | X | 0.02 |
| and Oral Los | | | | | | | |
| DMR Top | | | | | | | X |
| and Oral Veh | | | | | | | |

The lower mean collagen type IV intensity in the group that had Descemetorhexis and treatment with oral losartan alone compared to the group that had Descemetorhexis and treatment with topical vehicle and oral vehicle is likely due to one cornea with intense collagen type IV stromal levels in the latter group, as shown in the graph FIG. 3F, but the difference between these groups was not statistically significant. No effect of topical and/or oral losartan treatment on the levels of TGF beta-1 in the posterior stroma or at the posterior stromal surface compared to vehicle-treated corneas was noted, with considerable variability in the TGF beta-1 protein levels detected in the posterior stroma in all of the Descemetorhexis groups.

While the present disclosure is not limited to any particular mechanism, and an understanding of the mechanism is not necessary to practice the invention, it is believed that the main significance of the results shown in FIG. 3 indicates that the Losartan is penetrating into the cornea and blocking TGF beta, the main growth factor that stimulates fibrosis by driving the development of myofibroblasts. The TGF beta also stimulates the production of many collagens that are disordered in the cornea when myofibroblasts lay them down. Collagen type IV is just one collagen one can track well since in normal uninjured corneas it's only found in the epithelial basement membrane and Descemet's basement membrane, not in the stroma. One can see that in FIG. 3. Others like Collagen type 1 are also produced by the myofibroblasts. The myofibroblasts themselves and the disordered collagens they produce is the opacity in scarring fibrosis. So, it is believed that the topical Losartan inhibits the development of the myofibroblasts in the stroma and decreases their production of the scar collagens if some do develop.

The Descemetorhexis (DMR) model causes severe posterior stromal myofibroblast development and fibrosis in rabbits without visibly affecting the anatomy or function of the corneal epithelium (FIG. 1; FIG. 2; Medeiros et al., 2019; Sampaio et al., in press). The observation that topical losartan affected myofibroblast and fibrosis development in the posterior stroma of corneas that had DMR attests to the likelihood that topical losartan penetrated into the posterior corneal stroma in this study.

Collagen type IV is composed of six distinct alpha chains (α1 to α6) that assemble into heterotrimers (Pozzi et a., 2017). Virtually all basement membranes contain collagen type IV and the most ubiquitous trimer is composed of two α1 and one α2 chains. In the unwounded corneas, collagen type IV is prominent in the epithelial basement membrane (de Oliveria et al., in press) and Descemet's membrane (FIG. 3, and Sampaio et al., in press), but little collagen type IV is detected in stromal cells in the unwounded cornea (FIG. 3). A recent study showed that collagen type IV protein was produced by corneal fibroblasts and myofibroblasts in the posterior cornea after central Descemetorhexis and that TGF beta-1 upregulated collagen type IV mRNA production in corneal fibroblasts and myofibroblasts (Sampaio et al., in press). An important property of collagen type IV is that it directly binds TGF beta-1 and TGF beta-2 (Paralkar et al., 1991; Shibuya et al., 2006), as well as PDGF (Paralkar et al., 1991), and thereby decreases their binding to cognate receptors. While the present invention is not limited to any particular mechanism, it is believed that collagen type IV produced by corneal fibroblasts and myofibroblasts in the posterior cornea after Descemetorhexis functioned to downregulate TGF beta-1 and TGF beta-2 effects on the stromal cells in the area of production and to decrease more anterior stromal penetration of the TGF beta-1 and TGF beta-2. Thus, collagen type IV produced by corneal fibroblasts and myofibroblasts in the cornea may serve a negative feedback regulatory mechanism to modulate TGF beta effects on stromal cells.

In this Example, topical losartan treatment for one month after Descemetorhexis markedly down-regulated collagen type IV levels in the posterior stroma compared to vehicle except at the posterior corneal surface (FIG. 3). This showed that the topical losartan penetrated into the posterior corneal stroma and inhibited collagen type IV production by stromal cells in response to TGF beta-1, except at the posterior corneal surface that was devoid of Descemet's basement membrane and corneal endothelium at this time point at one month after Descemetorhexis where the localized stromal TGF beta-1 concentration was likely highest due to proximity to the TGF beta-1 reservoir in the aqueous humor. This observation provided direct evidence of topical losartan downregulation of TGF beta-modulated effects in the injured cornea—whether that TGF beta is derived from the aqueous humor and/or produced locally by stromal cells after injury (FIG. 3). The results in FIG. 3F show a trend for oral Losartan alone to possibly have some effect on posterior stromal collagen type IV levels at one month after Descemetorhexis compared to the topical vehicle and oral vehicle treated corneas, but the two groups were not statistically different and if a small difference were actually present it would be unlikely to be clinically meaningful. A corollary of this Example is that IHC and other assays that detect collagen type IV expression will allow the anti-TGF beta effects of losartan and other modulators to be monitored.

In conclusion, the present study suggests that topical losartan could be effective in the prophylactic prevention and treatment of corneal scarring fibrosis caused by trauma, infection, diseases and surgeries. Since myofibroblasts are dependent on TGF beta for survival (Wilson, 2020), the TGF beta-inhibitory effect of losartan could also be useful even for established corneal scars.

### REFERENCES FOR EXAMPLE 1

Bankhead, et al., 2017. QuPath: Open source software for digital pathology image analysis. Sci Rep. 7, 16878.
Cockerham and Hidayat, 1999. Retrocorneal membrane with myofibroblasts after perforating injury: an immunohistochemical and ultrastructural study of 11 cases. Cornea. 18, 700-6.
Cohn, et al., 2007. Angiotensin II type 1 receptor blockade attenuates TGF-[beta]-induced failure of muscle regeneration in multiple myopathic states. Nat. Med. 13, 204-10.
de Oliveira et al., (in press). Epithelial basement membrane regeneration after PRK-induced epithelial-stromal injury in rabbits: Fibrotic vs. non-fibrotic corneal healing. J. Ref. Surg., in press.
de Oliveira, et al., Exp. Eye Res. 202, 108325.
de Oliveira and Wilson, S. E., 2020. Biological effects of mitomycin C on late corneal haze stromal fibrosis following PRK. Exp Eye Res. 200, 108218.
Geirsson et al., 2012, 126, S189-97.
Ghosheh, et al., 2008., Eye Contact Lens. 34, 211-4.
Hindman, et al., 2019, Exp. Eye Res. 181, 49-60.
Jester, et al., 1997, Cornea. 16, 177-87.
Joung, et al., 2020, Int. J. Mol. Sci. 21, 2990.
Lavoie, et al., 2005, J. Hypertens. 23, 1895-1903
Lee, et al., 2001, J. Refract. Surg. 17, 334-41.
Lim, et al., 2001, Circulation. 103, 789-91.
Martínez-Garcia, et al., 2006, Exp. Eye Res. 83, 728-35.
Medeiros, et al. 2019, Vis. Sci. 60:1010-20.
Michel, et al., 2013, Pharmacol. Rev. 65, 809-48.
Mohan, et al., 2003, Exp. Eye Res. 76, 71-87.
Mohan, et al., 2008, Exp. Eye Res. 86, 235-40.
Møller-Pedersen, et al., 1998, Curr. Eye Res. 17, 736-47.
Netto et al., 2006, Exp. Eye Res. 82, 788-97.
Paralkar, et al., 1991, Dev. Biol. 143, 303-308.
Park, et al., 2012, Cell Transplant. 21, 2407-24.
Pozzi et al., 2017, Matrix Biol. 57, 58:1-11.
Sampaio, Exp. Eye Res., 2021 December; 213:108803.
Shibuya, et al., 2006, J. Dermatol. Sci. 41, 187-195.
Simpson, et al., 2000, Drugs Aging. 16, 227-50.
Srinivasan, 2012, Arch. Ophthalmol. 130, 143-50.
Wilson, 2019, J. Refract. Surg. 35, 506-16.
Wilson, 2020, Exp. Eye Res. 201, 108272.
Wilson, 2021, Invest. Ophth. Vis. Sci. 62, 8.
Wilson, 2021b, Exp. Eye Res. 207:108594.
Witcher, et al., 2001, Bull. World Health Organ. 79, 214-21.
Wylie-Sears, et al., 2014, et al., Biochem. Biophys. Res. Commun. 446, 870-5.
Yao et al., 2008, J. Huazhong Univ. Sci. Technolog. Med. Sci. 28, 543-8.

### Example 2

Topical Losartan and Corticosteroid Additively Inhibit Corneal Stromal Myofibroblast Generation and Scarring Fibrosis after Alkali Burn Injury

This Example describes evaluating the efficacy of losartan and prednisolone acetate in inhibiting corneal scarring fibrosis after alkali burn injury in rabbits. Briefly, sixteen New Zealand White rabbits were included in the study. Alkali burn injuries were produced using 1N NaOH on a 5 mm diameter Whatman #1 filter paper. Four corneas in each group were treated six times per day for one month with 50 μl of 1) 0.2 mg/ml losartan in BSS, 2) 1% prednisolone acetate, 3) combined 0.2 mg/ml losartan and 1% prednisolone acetate, or 4) BSS. Area of opacity and total opacity were analysed in standardized slit lamp photos with ImageJ. Corneas in both groups were cryofixed in OCT at one month after surgery and immunohistochemistry (IHC) was performed for alpha-SMA and keratocan or TGF beta-1 and collagen type IV. Treatment with combined topical losartan and prednisolone acetate significantly decreased slit lamp opacity area and intensity. This combination also markedly decreased stromal myofibroblast alpha-SMA area and intensity of staining per section and confined myofibroblasts to only the posterior stroma with repopulation of the anterior and mid stroma with keratocan-positive keratocytes after one month of treatment. Corneal fibroblasts produced collagen type IV not associated with BMs, especially in the anterior and posterior corneal stroma, and this production was decreased by topical losartan. In conclusion, combined topical losartan and prednisolone acetate was effective in decreasing myofibroblast-associated fibrosis after corneal alkali bums that produce full-thickness injury, including corneal endothelial damage.

### Materials and Methods

### Animals

All animal treatments and care were approved by the Institutional Animal Care and Use Committee (IACUC) at the Cleveland Clinic Foundation (Cleveland, OH, USA) and the Animal Care and Use Review Office of the Department of the Army (Fort Detrick, MD). All rabbits were treated in accordance with the tenets of the ARVO Statement for the Use of Animals in Ophthalmic and Vision Research. Sixteen 10- to 15-week-old female New Zealand White rabbits weighing 2.5 to 3.0 kg each were included in this example.

### Corneal Alkali Burn Method

Beginning twenty-four hours prior to alkali exposure and continuing for 5 to 7 days after treatment, rabbits received 60 ml children's liquid acetaminophen (Johnson and Johnson, Ft. Washington, PA) per 1 liter of drinking water. Prior to all alkali exposures and testing, the rabbits were anesthetized with 30 mg/kg ketamine hydrochloride and xylazine 5 mg/kg by IM injection. In addition, topical proparacaine hydrochloride 1% (Alcon, Ft Worth, TX, USA) was applied to each eye. As needed, due to signs of pain, rabbits also received 0.05 mg/kg buprenorphine by subcutaneous injection twice a day.

Alkali injuries were produced in one eye of rabbits with previously published methods¹⁰ using 1N sodium hydroxide (Sigma, St. Louis, MO) in balanced salt solution (BSS, 0.64% sodium chloride, 0.075% potassium chloride, 0.048% calcium chloride dihydrate, 0.03% magnesium chloride hexahydrate, 0.39% sodium acetate trihydrate, 0.17% sodium citrate dihydrate, pH 7.5, Alcon, Ft. Worth, TX) and a 5 mm diameter circular Whatman No. 1 filter paper (Cat #1001-6508, Fisher Scientific) wetted with 100 μl of 1N NaOH solution. Following the alkali burn injury, the cornea was irrigated profusely with BSS. Each injured cornea also received one drop of ciprofloxacin (Alcon, Ft. Worth, TX, USA) ten minutes after surgery and four times a day for one week and at least 15 minutes apart from the study medications.

Treatment with Topical Losartan and/or Prednisolone Acetate

Beginning immediately following alkali burn injury, four corneas in each group were treated six times per day (approximately 8 am, 10 am, 12 noon, 2 μm, 4 μm and 6 pm) for one month with 1) 50 μl of 0.2 mg/ml losartan (Merck & Co., Inc., Kenilworth, NJ) in BSS, 2) 50 μl of 1% prednisolone acetate (Alcon, Ft. Worth TX), 3) 50 μl of 0.2 mg/ml losartan in BSS and 50 μl of 1% prednisolone acetate (Alcon, Ft. Worth TX), at least 5 minutes apart, or 4) 50 μl of BSS.

Fluorescein Staining for Epithelial Defects at Two Weeks after Injury

At 2 weeks after alkali burn injury, topical 0.5% fluorescein in BSS was applied to each eye and the presence or absence of epithelial defect(s) was recorded.

### Standardized Slit Lamp Photographs, Corneal Angiography, and ImageJ Analysis of Corneal Opacity

At one month after sodium hydroxide exposure and treatments, with the rabbits under ketamine-xylazine general anesthesia, the eyes were dilated with two drops of 1% tropicamide (Akom Co., Lake Forest, IL) for thirty minutes. The study eye in each rabbit had slit lamp photographs with standardized illumination level and angle of illumination at 20× magnification with a Topcon (Oakland, NJ, USA) SL-D7 slit lamp photography system. For each study eye, the total area of opacity was determined by outlining the opacity with the freehand selection tool using ImageJ 1.53a analysis software calibrated to mm². The “raw internal density” in pixels for the opacified area in each cornea was also determined using ImageJ.

All corneas had fluorescent angiography at the peak of dye passage in the cornea immediately after injection of 1.5 ml of 10% sodium fluorescein (McKesson, Irvine, TX) in the central ear vein with the slit lamp system and digital camera system using a “barrier filter” that only transmitted 520-530 nm, the peak of fluorescein emission, with broad illumination of the cornea, as previously described.¹¹

### Corneal Fixation and Sectioning

One month after exposure and topical treatment, rabbits were euthanized after ketamine-xylazine general anesthesia with 100 mg/kg Beuthanasia (Shering-Plough, Kenilworth, NJ) by intravenous injection and bilateral pneumothorax. The corneo-scleral rims of eyes were removed with sharp Westcott scissors (Fairfield, CT, USA) and 0.12 forceps (Storz, St. Louis, MO). The cornea was centered in a 24×24×5 mm mold (Fisher Scientific, Pittsburgh, PA, USA) that was filled with OCT compound (Sakura Finetek, Torrance, CA, USA) and quick frozen on dry ice. Blocks were stored at −80° C. until sectioning.

OCT blocks were bisected at the center of the cornea and 8 μm thick transverse sections were cut from the central cornea with a cryostat (HM 505M; Micron GmbH, Walldorf, Germany). Three sections from each cornea were placed on each 25 mm×75 mm×1 mm Superfrost Plus microscope slide (Fisher Scientific, Pittsburgh, PA, USA). Slides with sections were maintained at −20° C. prior to immunohistochemistry.

### Immunohistochemistry and Fibrotic Area Opacity Intensity Analysis

Multiplex immunohistochemistry (IHC) was performed using previously described methods⁸ and primary antibodies (Table 7) confirmed by western blotting and IHC to recognize rabbit antigens or isotypic control antibodies (Thermo Fisher Scientific, Waltham, MA), and secondary fluorescent tagged antibodies (Table 7).

TABLE 7
Primary and secondary antibodies

| PRIMARY | | | Ab | | |
|---|---|---|---|---|---|
| Antigen | Company | Species | isotype | Catalog # | Dilution |
| Keratocan | Winston | Goat | IgG | — | 1:200 |
| | Kao | | | | |
| SMA | Dako | Mouse | IgG₂ₐ | M0851 | 1:400 |
| TGF beta-1 | Genetex | Mouse | IgG₁ | GTX21279 | 1:100 |
| Collagen type | Millipore | Goat | IgG1 | AB769 | 1:1200 |
| IV | | | | | |

| SECONDARY | | | | | |
|---|---|---|---|---|---|
| Antibody | Company | Species | Isotype | Catalog # | Dilution |
| Alexa Fluor 488 | TFS^{#} | donkey | IgG | A21202 | 1:200 |
| anti-mouse | | | | | |
| Alexa Fluor 488 | TSF | donkey | IgG | A11055 | 1:200 |
| anti-goat | | | | | |
| Alexa Fluor 568 | TSF | donkey | IgG | A10037 | 1:200 |
| anti-mouse | | | | | |
| Alexa Fluor 568 | TSF | donkey | IgG | A11057 | 1:200 |
| anti-goat | | | | | |

^{#}TSF is Thermo Fisher Scientific

The collagen type IV antibody (Cat. #AB769, Millipore, Temecula CA) was raised against purified human and bovine collagen type IV that had been affinity purified with human and bovine collagen type IV crosslinked to agarose and cross-absorbed by the manufacturer with human and bovine collagens type I, II, III, V and VI to eliminate cross-reactivity. This collagen type IV antibody was shown previously to bind rabbit collagen IV in IHC,^{8,9} and recognizes the alpha-1/alpha-2 chains but not the alpha-3 to alpha-6 chains. The keratocan antibody is raised against peptide H2N-LRLDGNEIKPPIPIDLVAC-OH (SEQ ID NO:1). This marker was used to identify keratocytes in situ. The TGF beta-1 antibody (GeneTex, Irvine, CA) binds rabbit TGF beta-1 in IHC and shows no reactivity to TGF beta-2 or TGF beta-3.⁵

The area of the α-SMA-positive stroma in mm² and the total α-SMA opacity in pixels were quantitated using standardized images obtained with a 10× objective on a Leica DM6B upright microscope equipped with an automated stage and Leica 7000 T camera using the LAS X software (Leica Microsystems, GmbH, Wetzlar, Germany). The means of three central corneal sections were analyzed from each cornea to provide the area of α-SMA-positive stroma and the total α-SMA opacity. The area of α-SMA-positive stroma in mm² for each cornea was determined from full diameter and thickness central corneal section images (all converted to 300 DPI, 885 width×500 height, files with identical+50% brightness increase in Photoshop for all images) with the ImageJ 1.53a analysis software using the freehand selection tool to delineate the area(s) of α-SMA-positive staining. In some corneas, two or more separated areas were present, and the summation of these areas was used as the value for that cornea. In these same sections for each cornea, the total α-SMA-positive intensity in pixels was also determined with ImageJ, similarly by using the summation if separate α-SMA-positive areas were present.

All corneas in each group had IHC for collagen type IV. Images from each cornea were converted to uniform 300 DPI, 875×568 pixel, files. Each cornea had three measurements of signal intensity in three randomly positioned 100×50 ImageJ analysis rectangles in both the anterior cornea (anterior side of rectangle at the anterior stromal surface posterior to the EBM, if present) and posterior cornea (posterior side of rectangle at the posterior stromal surface anterior to Descemet's membrane, if present). The staining intensity within each box was determined with the analyze histogram function and the mean of three boxes was the intensity value in the anterior or posterior stroma for that cornea.

### Statistics

Statistical analyses were performed using the Kruskal-Wallis test followed by post-hoc Dunn's-Bonferroni test and p<0.05 was considered statistically significant.

### Results

Persistent Epithelial Defects after Alkali Burn

At two and four weeks after the alkali burn, all corneas in all groups had at least a 1 mm diameter epithelial defect, and no differences between the treatment groups were found.

Slit Lamp Stromal Opacity and Central Corneal Neovascularization (CNV) after Alkali Burn

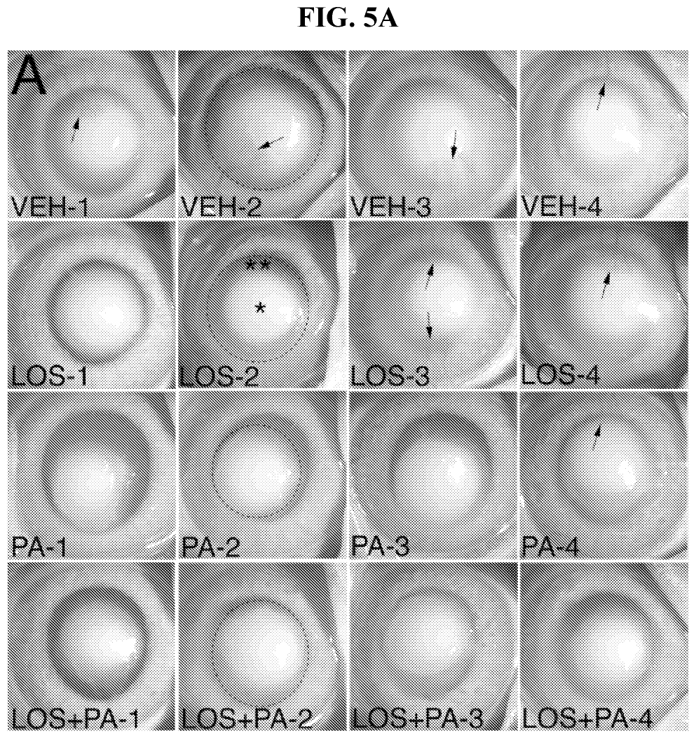

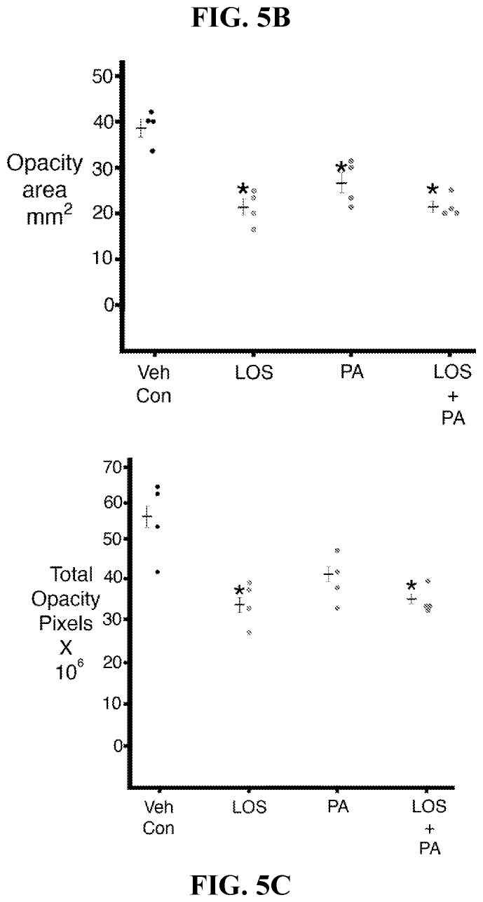

Using the 100 μl 1N NaOH on a 5 mm filter paper circle for 1 min method, followed by treatment with topical 0.2 mg/ml losartan, 1% prednisolone acetate, 0.2 mg/ml losartan and 1% prednisolone acetate, or BSS vehicle, six times per day for one-month, central stromal opacity of each of the corneas was as shown in FIG. 5A. Examples of ImageJ delineations used to measure total stromal opacity area are shown in one cornea for each treatment. Notice that these quantitation areas contained a much denser center (* in LOS-2, for example) and a less dense periphery (** in LOS-2, for example) for each imaged cornea. The total area of opacity in mm² for each cornea in each group is shown in FIG. 5B. Statistical comparisons between the groups are shown in Table 8.

TABLE 8

| | | | | Losartan + |
|---|---|---|---|---|
| | Vehicle | | Prednisolone | Prednisolone |
| | control | Losartan | Acetate | acetate |
| Vehicle | X | 0.02 | 0.02 | 0.02 |
| Control | | | | |
| Losartan | | X | 0.19 | 0.88 |
| Prednisolone | | | X | 0.08 |
| Acetate | | | | |
| Losartan + | | | | X |
| Prednisolone | | | | |
| acetate | | | | |

The losartan alone, prednisolone alone, and combined losartan and prednisolone groups were significantly different from the BSS vehicle group, but not significantly different from each other. ImageJ was also used to determine the total opacity intensity in pixels for the combined dense central and less dense peripheral area in each cornea (as indicated by the dashed line in one cornea from each group) and that data is shown in FIG. 5C. Statistical comparisons between the groups are shown in Table 9.

TABLE 9

| | | | | Losartan + |
|---|---|---|---|---|
| | Vehicle | | Prednisolone | Prednisolone |
| | Control | Losartan | Acetate | acetate |
| Vehicle | X | 0.02 | 0.08 | 0.02 |
| Control | | | | |
| Losartan | | X | 0.15 | 0.77 |
| Prednisolone | | | X | 0.08 |
| Acetate | | | | |
| Losartan + | | | | X |
| Prednisolone | | | | |
| acetate | | | | |

The losartan alone and combined losartan and prednisolone acetate groups were significantly different from the BSS vehicle group, but not significantly different from each other. The difference between the 1% prednisolone acetate group and the BSS vehicle group did not reach statistical significance. The combined losartan and prednisolone acetate group, however, had the lowest standard error of the mean for both stromal opacity area and total opacity intensity.

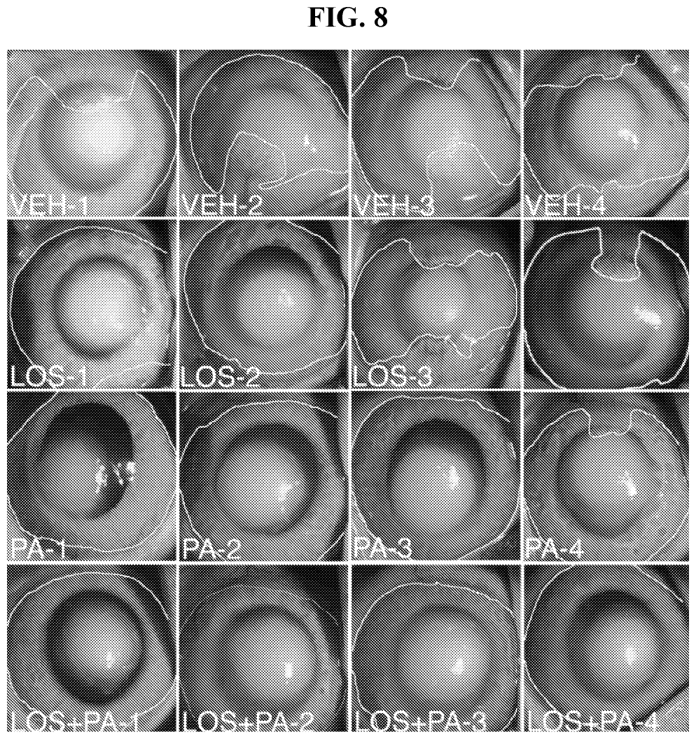

Central corneal neovascularization (FIG. 8) developed in all four BSS vehicle-treated corneas, in two corneas treated with topical 0.2 mg/ml losartan alone, and in one of the corneas treated with 1% prednisolone acetate alone, and in none of the corneas treated with both 0.2 mg/ml losartan and 1% prednisolone acetate.

### IHC for Keratocan-Positive Keratocytes and α-SMA-Positive Myofibroblasts

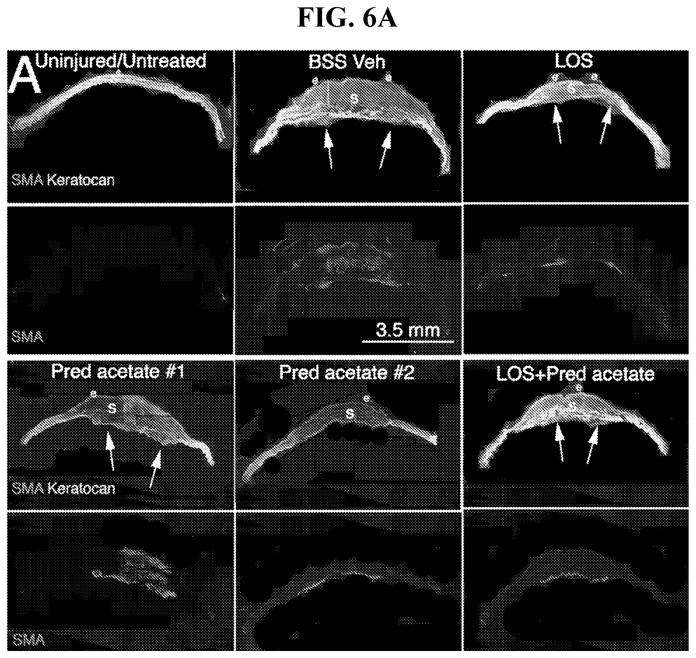

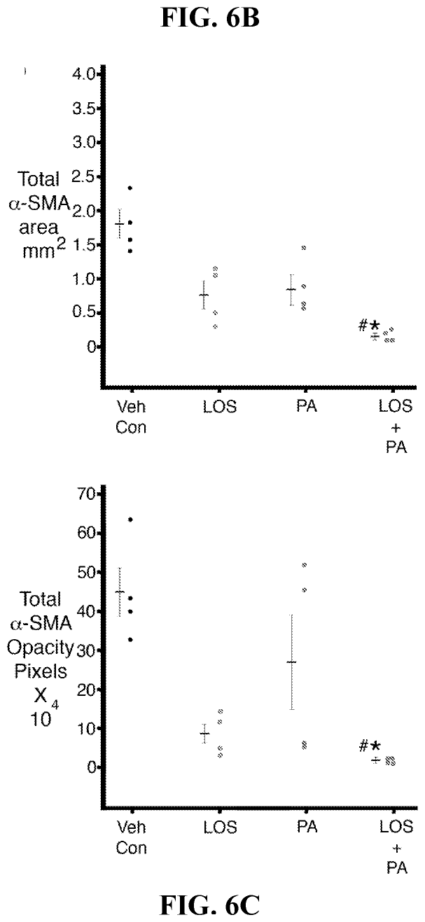

FIG. 6A shows representative central sections from corneas in each group. All corneas with alkali injury in this study had no corneal endothelium within the central 6 to 10 mm at one month after injury. No α-SMA-positive myofibroblasts were found in any uninjured cornea. All corneas treated with BSS vehicle had full thickness or nearly full thickness α-SMA-positive myofibroblasts, although some patches of keratocan-positive keratocytes were present, as shown in FIG. 6A. In corneas treated with losartan alone, α-SMA-positive myofibroblasts were confined mostly to the posterior half of the stroma in all corneas, as shown in the example in FIG. 6A. The α-SMA-positive myofibroblast localization tended to be more variable in the prednisolone acetate group. In two corneas in that group, the α-SMA-positive myofibroblasts were found throughout the stroma (as shown in example #1 in FIG. 6A) and in two corneas the α-SMA-positive myofibroblasts were present only in the posterior half of the stroma (as shown in example #2 in FIG. 6A). In all four corneas in the losartan+prednisolone acetate combined group, the α-SMA-positive myofibroblasts were confined to the far posterior stroma, with repopulation of the more anterior stroma with keratocan-positive keratocytes (as shown in FIG. 6A).

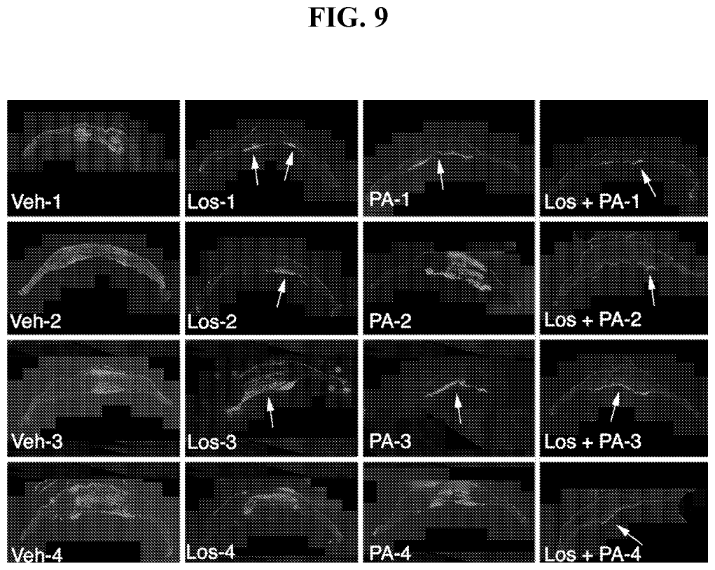

FIG. 9 shows α-SMA-positive staining in a representative section from each cornea that was used for ImageJ analysis of the total area of stromal α-SMA-positive staining and the total α-SMA-positive opacity. The variability of stroma α-SMA-positive staining in the prednisolone acetate group can be noted in this figure. ImageJ analysis results for the total α-SMA area in each cornea is shown in FIG. 6B. Table 10 shows the statistical comparisons between the groups.

TABLE 10

| | | | | Losartan + |
|---|---|---|---|---|
| | Vehicle | | Prednisolone | Prednisolone |
| | control | Losartan | acetate | acetate |
| Vehicle | X | 0.06 | 0.14 | 0.0005 |
| Control | | | | |
| Losartan | | X | 0.71 | 0.10 |
| Prednisolone | | | X | 0.04 |
| Acetate | | | | |
| Losartan + | | | | X |
| Prednisolone | | | | |
| acetate | | | | |

The combined losartan-prednisolone acetate group had significantly less α-SMA area than the vehicle control group. The combined losartan-prednisolone acetate group also had significantly less α-SMA area than the prednisolone acetate group. Other differences did not reach statistical significance, although it can be noted there was a trend towards the losartan group being significantly different from the vehicle control group. ImageJ analysis results for the total α-SMA opacity intensity in pixels for each cornea is shown in FIG. 6C. Table 11 shows the statistical comparisons between the groups.

TABLE 11

| | | | | Losartan + |
|---|---|---|---|---|
| | Vehicle | | Prednisolone | Prednisolone |
| | control | Losartan | acetate | acetate |
| Vehicle | X | 0.10 | 0.55 | 0.002 |
| Control | | | | |
| Losartan | | X | 0.29 | 0.14 |
| Prednisolone | | | X | 0.01 |
| acetate | | | | |
| Losartan + | | | | X |
| Prednisolone | | | | |
| acetate | | | | |

Again, the combined losartan-prednisolone acetate group had highly significantly less α-SMA opacity intensity in pixels than the vehicle control group. Also, the combined losartan-prednisolone acetate group was significantly less than the prednisolone acetate alone group in total α-SMA opacity intensity. Other differences did not reach statistical significance. Importantly, note the low variability for both the total α-SMA area (FIG. 6B) and the total α-SMA opacity intensity (FIG. 6C) in the combined losartan+prednisolone acetate group.

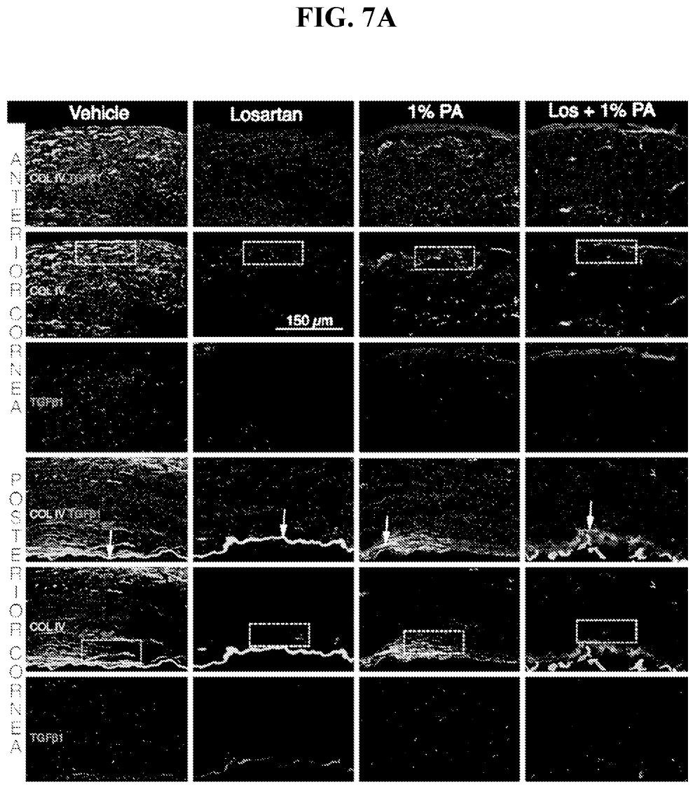

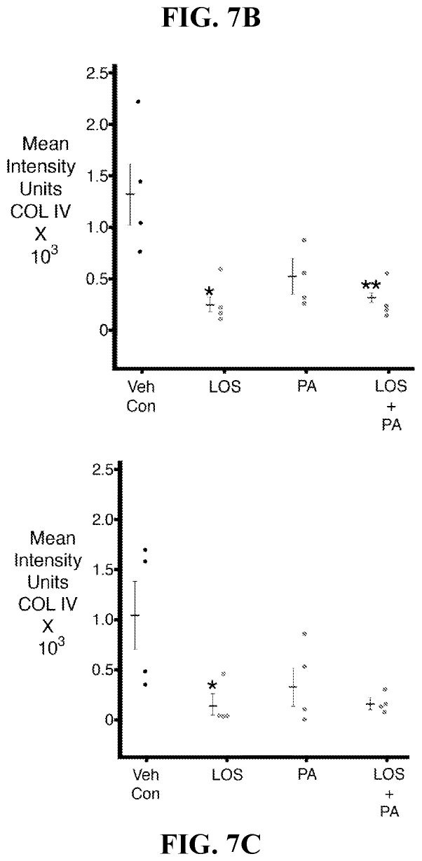

FIG. 7A shows representative duplex IHC for collagen type IV and TGF beta-1 in the anterior and posterior stroma for representative corneas in each group and also shows example ImageJ quantitation rectangles. FIG. 7B shows quantitation for COL IV staining intensity in the anterior stroma of the corneas in each group. Both losartan treatment and losartan+1% prednisolone acetate significantly decreased the mean intensity units of collagen type IV in the anterior stroma compared to vehicle treatment. Prednisolone acetate alone produced a trend towards a decrease in collagen type IV staining intensity in the anterior stroma compared to vehicle treatment, but the difference did not reach statistical significance. Table 12 shows the statistical comparisons for collagen type IV intensity in the anterior stroma between the groups.

TABLE 12

| | | | | Losartan + |
|---|---|---|---|---|
| | Vehicle | | Prednisolone | Prednisolone |
| | control | Losartan | acetate | acetate |
| Vehicle | X | 0.004 | 0.16 | 0.01 |
| Control | | | | |
| Losartan | | X | 0.14 | 0.71 |
| Prednisolone | | | X | 0.27 |
| Acetate | | | | |
| Losartan + | | | | X |
| Prednisolone | | | | |
| acetate | | | | |

FIG. 7C shows quantitation for COL IV staining intensity in the posterior stroma of the corneas in each group. Only the topical losartan treatment was significantly different from the vehicle treatment, although the combined losartan+1% prednisolone acetate group trended towards significance. Table 13 shows the statistical comparisons for collagen type IV intensity in the anterior posterior between the groups.

TABLE 13

| | | | | Losartan + |
|---|---|---|---|---|
| | Vehicle | | Prednisolone | Prednisolone |
| | control | Losartan | acetate | acetate |
| Vehicle | X | 0.01 | 0.15 | 0.07 |
| Control | | | | |
| Losartan | | X | 0.30 | 0.50 |
| Prednisolone | | | X | 0.71 |
| Acetate | | | | |
| Losartan + | | | | X |
| Prednisolone | | | | |
| acetate | | | | |

Chemical injuries to the cornea caused by sodium hydroxide (NaOH) vary from mild, self-limited ocular surface disturbances to devastating burns affecting the corneal epithelium, corneal limbus, stroma, and the corneal endothelium.^{10, 12-17} Severe alkali burn injuries are frequently associated with corneal neovascularization (CNV) and persistent epithelial defects.¹²⁻¹⁷ Severe NaOH corneal burns can also injure the trabecular meshwork, iris, ciliary body, lens, retina, and optic nerve.¹²

The 1N NaOH corneal burn injury method used in the present example was used in many prior rabbit studies.^{10, 13, 14} The one-month time point for analysis of the effect of alkali burn injury and the potential effects of the topical medications was selected because one-month was when the wound healing response to injury to the cornea peaked in prior studies.²⁻⁶ The current example showed that severe chemical injury with 100 microliters of 1N NaOH delivered using a 5 mm filter paper delivery system for one minute penetrated through the stroma and uniformly injured a large underlying area of the corneal endothelium, and often Descemet's membrane, approximately 8 to 10 mm in diameter. No evidence of limbal injury was noted using this method. Similarly, no evidence of iris or lens damage was noted in the rabbit eyes after this injury. In certain experiments, even 15 seconds of exposure to 1N NaOH using this method damaged the corneal endothelium (Sampaio LP and Wilson S E, unpublished data, 2021), and, therefore, dilutions of the NaOH would likely be needed to produce a model with injury confined to the epithelium and anterior stroma of the cornea.

The mode of cell death of the affected epithelium, keratocytes, and corneal endothelium produced by the 1N NaOH was previously reported to be necrosis.^{15,16} Cellular necrosis, along with denaturation of the underlying collagen fibrils,¹⁷ likely triggered the severe corneal inflammatory response that was noted with the slit lamp in all corneas in this study during the first few days to two weeks after injury.

The opacities remaining at one month after injury and treatment in all groups were characterized by a dense central zone surrounded by a less dense ring (FIG. 5A). It was believed that the dense central area represents denatured and disorganized collagen fibrils produced by the original NaOH injury, along with myofibroblasts that developed and the large amounts of disordered extracellular matrix these fibrotic cells produced.^{5, 6, 18} The less dense ring may be associated with less severely damaged stromal collagen and corneal fibroblasts, along with lesser amounts of disordered extracellular matrix produced by corneal fibroblasts.

Losartan is an angiotensin converting enzyme (ACE) II receptor antagonist that also inhibits TGF beta signaling.^{9, 19-24} In the present Example, topical treatment with 0.2 mg/ml losartan in BSS, 1% prednisolone acetate, or combined losartan and prednisolone acetate six times per day decreased the total corneal opacity area measured with ImageJ on the standardized slit lamp images (FIG. 5B). The differences in the total area of opacity were not significantly different between the losartan, prednisolone acetate, or combined losartan/prednisolone acetate groups (Table 8). The total opacity in pixels in the opacified area of cornea, also measured with ImageJ, was significantly lower in the 0.2 mg/ml losartan group or the combined 0.2 mg/ml losartan+1% prednisolone acetate group compared to the vehicle BSS group. The 1% prednisolone acetate alone group trended towards decreased total opacity compared to the vehicle BSS group, but the difference did not reach statistical significance (Table 9).

One of the most interesting findings in this Example relates to myofibroblast development and stromal fibrosis in the different treatment groups (FIG. 6A). All corneas that had the alkali burn followed by treatment with vehicle BSS had α-SMA-positive myofibroblasts and fibrosis throughout the full thickness of the cornea, although this fibrosis appeared to be greatest adjacent to the anterior and posterior stromal surfaces (FIG. 9), likely due to higher concentrations of TGF beta-1 and TGF beta-2 penetrating the stroma from the tears, epithelium, residual peripheral corneal endothelium and aqueous humor at the corneal surfaces.^{5, 6} In the NaOH-injured corneas treated with topical 0.2 mg/ml losartan, the greatest density of α-SMA-positive myofibroblasts tended to be noted in the posterior half of the stroma, although lesser amounts of anterior stromal α-SMA-positive myofibroblasts were noted in the two losartan-treated corneas after one month of treatment (FIG. 9). Persistent corneal epithelial defects are themselves associated with the development of anterior stromal myofibroblasts and fibrosis²⁵ and could have had a role in anterior myofibroblasts noted in two losartan-treated corneas. Alkali-injured corneas treated with 1% prednisolone acetate alone were more variable in stromal myofibroblast development (FIG. 9). After injury and treatment with combined losartan and prednisolone acetate, however, the α-SMA-positive myofibroblasts in all four corneas were restricted to the far posterior stroma (FIG. 9). It is important to note that the corneal endothelium and Descemet's membrane had not regenerated in any cornea in any of the treatment groups by the one-month time point, as can be noted in the representative corneas in FIG. 6A.

When the area of α-SMA-positive myofibroblasts was determined using ImageJ in each of the central corneas (FIG. 6B), the combined losartan+prednisolone acetate group was significantly different from the vehicle BSS-treated group (p=0.0005, Table 10). This combined losartan+prednisolone acetate group also had low standard error of the mean for the area of α-SMA staining (FIG. 6B). The combined losartan and prednisolone acetate group had significantly lower area of α-SMA staining than the prednisolone acetate alone group. Similarly, when the total α-SMA intensity per corneal section was determined using ImageJ in each of the corneas (FIG. 6C), the combined losartan+prednisolone acetate treatment group was significantly lower than the vehicle BSS-treated group (p=0.002, Table 11) and the combined losartan+prednisolone acetate treatment group was significantly lower than the prednisolone acetate alone group (p=0.01). While the present invention is not limited to any particular mechanism and an understanding of the mechanism is not necessary to practice the invention, we hypothesize that the efficacy of the combined losartan+prednisolone acetate treatment after the severe alkali burns was attributable to the corticosteroid modulation of inflammation due to the severe tissue necrosis and the losartan modulation of pro-fibrotic TGF beta effects on stromal myofibroblast development and, therefore, disordered collagen production by these cells. Bone marrow-derived fibrocytes also enter the stroma from the limbus after injury and, in addition to corneal fibroblasts, are TGF beta-driven precursors to myofibroblasts.^{26, 27} Corticosteroids inhibit the proliferation of fibrocytes necessary for generation of large numbers of myofibroblasts²⁸ and also trigger fibrocyte apoptosis.²⁹ Thus, the topical corticosteroids could contribute to losartan inhibition of myofibroblast development from both corneal fibroblasts and fibrocytes via these mechanisms.

This Example demonstrated that severe sodium hydroxide injuries are commonly associated with damage to the corneal endothelium and Descemet's membrane that increase the corneal fibrosis response. This is analogous to findings regarding the effects of chemical burns caused by bioweapon agents, such as sulfur mustard, where corneal endothelial damage was a major determinate of the long-term outcomes of injury.^{30,31} Combined topical losartan and corticosteroids could also decrease myofibroblast generation and corneal scarring fibrosis that occur in response to these chemical bio-weapon agents.

### REFERENCES FOR EXAMPLE 2

1. Witcher et al., Bull World Health Organ. 2001; 79:214-21.
2. Torricelli et al., Invest Ophth Vis Sci. 2013:54:4026-33.
3. Marino et al., J Ref Surg. 2017; 33:337-346.
4. Marino et al., Exp Eye Res. 2017; 161:101-105.
5. de Oliveira et al., Exp Eye Res. 2021; 202:108325
6. de Oliveira et al., J Ref Surg. 2022; 38:50-60.
7. Medeiros et al., Invest Ophthalmol Vis Sci. 2018; 59:4044-4053.
8. Sampaio et al., Exp Eye Res. 2021; 213:108803.
9. Sampaio et al., Exp Eye Res. 216:108940.
10. Ishizaki et al., Invest Ophthalmol Vis Sci. 1993; 34:3320-8.
11. Nirankari et al., Ophthalmology 1993; 100:111-8.
12. Paschalis et al., J Pathol. 2017; 187:1327-1342.
13. Lee et al., Graefes Arch Clin Exp Ophthalmol. 2014; 252:951-61.
14. Burns et al., Invest Ophthalmol Vis Sci. 1989; 30:1569-75.
15. Yi et al., Trans Ophthalmol Soc U K. 1978; 98:379-82.
17. Maskati et al., Ind J Ophthalmol. 1987; 35:396-400.
18. Jester et al., J Cell Sci. 1999; 112:613-22.
19. Wylie-Sears et al., Biochem Biophys Res Commun. 2014; 446: 870-875.
20. Geirsson et al., Circulation. 2012; 126 (11 Suppl 1):S189-197.
21. Park et al., Cell Transplant. 2012; 21:2407-2424.
22. Lim et al., Circulation. 2001; 103:789-791.
23. Lavoie et al., J Hypertens. 2005; 23:1895-1903.
24. Cohn et al., Nat Med. 2007; 13:204-210.
25. Wilson et al., J Ref Surg. 2018; 34:59-64.
26. Lassance et al., Exp Eye Res. 2018; 170:177-187.
27. de Oliveira et al., Invest Ophthalmol Vis Sci. 2020; 61:28-35.
28. Hayashi et al., Biomed Res Int. 2014; 2014:738625.
29. Lo et al., J Allergy Clin Immunol. 2015; 135:1186-95.e1-6.
30. McNutt et al., Invest Ophthalmol Vis Sci. 2013; 54:6735-44.
31. McNutt et al., Cornea. 2020; 39:640-648.
32. Paralkar et al., Dev. Biol. 1991; 143:303-308.
33. Shibuya et al., J Dermatol Sci. 2006; 41:187-195.
34. Wilson et al., Matrix Bio. 2022; 109:162-172

### Example 3

Losartan Inhibition of Myofibroblast Generation and Late Haze (Scarring Fibrosis) after Photorefractive Keratectomy (PRK) in Rabbits

This Example describes evaluating the effect of topical losartan compared to vehicle on the generation of myofibroblasts and development of late haze scarring fibrosis after PRK in rabbits.

### SUMMARY

Briefly, rabbits (12) had −9D PRK in one eye followed by 50 μl of topical 0.2 mg/ml losartan or 50 μl of vehicle six times per day for one month. Standardized slit lamp photos were obtained prior to euthanasia. Duplex IHC was performed on cryofixed corneas for myofibroblast marker alpha-smooth muscle actin (α-SMA) and keratocyte marker keratocan or collagen type IV and transforming growth factor (TGF) beta-1. ImageJ was utilized for quantitation. Topical losartan compared to vehicle significantly decreased corneal opacity (P=0.04) and significantly decreased anterior stromal myofibroblast generation (P=0.01) at one month after PRK. Topical losartan compared to vehicle also decreased anterior stromal non-basement membrane collagen type IV at one month after PRK (P=0.004). Topical ACEII receptor inhibitor losartan, a known inhibitor of TGF beta signaling, decreased late haze scarring fibrosis and myofibroblast generation after −9D PRK in rabbits compared to vehicle. It also decreases TGF beta-modulated, corneal fibroblast-produced, non-basement membrane stromal collagen type IV.

### BACKGROUND

Clinically significant late corneal haze (also termed corneal stromal scarring fibrosis) continues to be reported as a complication after photorefractive keratectomy (PRK). (1) The incidence of late haze after PRK decreased markedly after widespread adoption of single-dose intraoperative mitomycin C, (2,3) but continues to be noted in some eyes despite mitomycin C application and is then termed “breakthrough haze”. (4). Although late haze is most commonly noted after moderate to high myopia or hyperopia corrections with PRK, it occasionally is noted after PRK corrections for low myopia, especially when mitomycin C intraoperative treatment is omitted or when there is a persistent epithelial defect following surgery. (2,3).

Several investigations in rabbits have demonstrated that defective regeneration of the epithelial basement membrane (EBM) and the development of subepithelial myofibroblasts 10 underlie the development of late haze corneal stromal fibrosis after PRK. (5-7). Myofibroblasts develop in the cornea from both keratocyte-derived corneal fibroblasts and bone marrow-derived fibrocytes via a cellular developmental program driven primarily by transforming growth factor (TGF) beta-1 and TGF beta-2.(8,9). TGF beta-1 and TGF beta-2 continuously enter the stroma from the corneal epithelium and tears after PRK when there is delayed regeneration of the EBM that modulates TGF beta passage into the stroma, along with the apical epithelial barrier function. (6, 10-12). Perlecan and collagen type IV are EBM components that serve as gatekeepers regulating passage of TGF beta-1 and TGF beta-2 into the stroma. (10-12). Myofibroblasts are critically dependent on an adequate and ongoing source of TGF beta-1 and/or TGF beta-2 for full development and survival, and these fibrotic cells and their precursor cells undergo apoptosis when deprived of signaling by these growth factors. (8,9).

### Materials and Methods

### Animals and Surgery

Animal procedures were approved by the Institutional Animal Care and Use Committee at the Cleveland Clinic Foundation and animals were treated in accordance with the tenets of the Association for Research in Vision and Ophthalmology Statement for the Use of Animals in Ophthalmic and Vision Research. Twelve female 12- to 15-week-old New Zealand white rabbits weighing 2.5 to 3 kg each were included. Starting 24 hours prior to surgery and continuing 5 days after PRK, all rabbits received 60 mL children's liquid acetaminophen (Johnson & Johnson, Ft. Washington, PA, USA) per 1 L of drinking water. One eye of each rabbit was randomly selected to have −9.0 D (on myopia scale) PRK and received 2 drops of topical 1% proparacaine hydrochloride (Alcon, Fort Worth, TX, USA) prior to the surgery. PRK with manual epithelial debridement using a #6400 Beaver blade (MedexSupply, Passaic, NJ) was performed using previously published methods^{6, 7, 10} with a VISX (Santa Clara, CA) S4 IR excimer laser while the rabbit was under general anesthesia with 30 mg/kg ketamine hydrochloride and xylazine 5 mg/kg by intramuscular (IM) injection and topical anesthesia with 1% proparacaine (Alcon, Ft. Worth, TX).⁶ The opposite cornea was included as an unwounded control since no contralateral effects of PRK have been noted in prior studies.^{5, 6, 7, 10}

### Medications

Beginning immediately after surgery, six eyes that had PRK were treated with 50 μl of balanced salt solution (BSS, 0.64% sodium chloride, 0.075% potassium chloride, 0.048% calcium chloride dihydrate, 0.03% magnesium chloride hexahydrate, 0.39% sodium acetate trihydrate, 0.17% sodium citrate dihydrate, pH 7.5) six times a day (approximately 8 am, 10 am, 12 noon, 2 pm, 4 pm and 6 pm) and six eyes that had PRK were treated with 50 μl of 0.2 mg/ml losartan (Merck & Co., Inc., Kenilworth, NJ, USA) in BSS six times a day. Treatment with vehicle or losartan continued for one month after the PRK surgery. Eyes that had PRK were also treated with one drop of topical ciprofloxacin three times a day at least 5 minutes from the other topical medication until the epithelium had closed (the epithelium in all eyes closed by 5 days after surgery). No corticosteroids were administered in either group.

### Standardized Slit-Lamp Photographs and ImageJ Measurement of Corneal Opacity

At 1 month after PRK and treatment, each rabbit was placed under ketamine-xylazine general anesthesia and the eyes were dilated with two drops of 1% tropicamide (Akorn Co., Lake Forest, IL, USA) for 30 minutes. The study eye in each rabbit had slit-lamp photographs with standardized illumination level and angle of illumination at 20× magnification with a Topcon (Oakland, NJ, USA) SL-D7 slit-lamp photography system. For each study cornea, the mean opacity in pixels in the central 3.5 mm of the PRK ablated zone was determined by using ImageJ 1.53a analysis software (National Institutes of Health, Bethesda, MD, USA).

### Corneal Cryo-Fixation and Sectioning

Rabbits were euthanized while under ketamine-xylazine general anesthesia with 100 mg/kg Beuthanasia (Shering-Plough, Kenilworth, NJ, USA) by intravenous injection followed by bilateral pneumothorax. The corneo-scleral rims were removed with sharp Westcott scissors (Fairfield, CT, USA) and 0.12 forceps (Storz, St Louis, MO, USA). The corneo-scleral rim was then centered in a 24-mm×24-mm×5-mm mold (Fisher Scientific, Pittsburgh, PA, USA) that was filled with optimal cutting temperature (OCT) compound (Sakura Finetek, Torrance, CA, USA) and quick frozen on dry ice. Blocks were stored at −80° C. until sectioning. Blocks were bisected at the center of the cornea and 10-pm-thick transverse sections were cut from the central cornea with a cryostat (HM 505M; Micron 5 GmbH, Walldorf, Germany). Three sections from each cornea were placed on each 25-mm×75-mm×1-mm Superfrost Plus microscope slide (Fisher Scientific). Slides with sections were maintained at −20° C. prior to immunohistochemistry (IHC).

### Immunohistochemistry (IHC) and Quantitation of Stromal Alpha-Smooth Muscle Actin and Collagen Type IV

Duplex IHC for: 1) alpha-smooth muscle actin and keratocan or 2) collagen type IV and TGF beta-1 was performed using previously described methods and primary antibodies confirmed by Western blotting and IHC to recognize rabbit antigens or isotypic non-specific control antibodies (ThermoFisher Scientific, Waltham, MA, USA) and previously described secondary fluorescent tagged antibodies (Table 14).²⁰

TABLE 14
Primary and secondary antibodies

| PRIMARY | | | | | |
|---|---|---|---|---|---|
| antibodies | Company/ | | Ab | | |
| Antigen | source | Species | isotype | Catalog # | Dilution |
| Keratocan | Winston | goat | IgG | — | 1:200 |
| | Kao | | | | |
| α-SMA | Dako | mouse | IgG₂ₐ | M0851 | 1:400 |
| TGF beta-1 | Genetex | mouse | IgG₁ | GTX21279 | 1:100 |
| Collagen type | Millipore | goat | IgG1 | AB769 | 1:2000 |
| IV | | | | | |

| SECONDARY | | | | | |
|---|---|---|---|---|---|
| antibodies | | | | | |
| Antibody | Company | Species | Isotype | Catalog # | Dilution |
| Alexa Fluor 488 | TFS^{#} | donkey | IgG | A21202 | 1:200 |
| anti-mouse | | | | | |
| Alexa Fluor 488 | TFS | donkey | IgG | A11055 | 1:200 |
| anti-goat | | | | | |
| Alexa Fluor 568 | TFS | donkey | IgG | A10037 | 1:200 |
| anti-mouse | | | | | |
| Alexa Fluor 568 | TFS | donkey | IgG | A11057 | 1:200 |
| anti-goat | | | | | |

^{#}TSF is Thermo Fisher Scientific

The collagen type IV antibody (cat. AB769; Millipore, Temecula, CA, USA) was generated against purified human and bovine collagen type IV (affinity purified with human and bovine collagen type IV crosslinked to agarose and then cross-absorbed by the manufacturer with human and bovine collagens type I, II, III, V, and VI to eliminate cross-reactivity). This collagen type IV antibody was shown previously to bind rabbit collagen IV in IHC^{6, 20} and binds the α-1/α-2 chains but not the α-3 to α-6 chains.

The keratocyte-specific keratocan antibody raised against peptide H2N-LRLDGNEIKPPIPIDLVAC-OH (SEQ ID NO:1). The TGF beta-1 antibody (GeneTex, Irvine, CA, USA) that was used binds rabbit TGF beta-1 in IHC and shows no reactivity to TGF-β2 or TGF-β3.^{6, 20} Images were obtained at 100× total magnification on a Leica DM6B upright microscope equipped with an automated stage and Leica 7000 T camera using the LASX software (Leica Microsystems, GmbH, Wetzlar, Germany).

All images were converted to 300 DPI 900 pixel width×672 pixel height images with Photoshop 22.1.1 (Adobe, San Jose, CA). The mean pixels of stromal α-SMA or stromal collagen type IV were determined in a 900-pixel wide×235-pixel high rectangle (for either α-SMA or collagen type IV quantitation) with ImageJ in three sections for each cornea using the image panels showing only the color of interest. The mean from three corneal sections was used as the value for stromal α-SMA or stromal collagen type IV for each individual cornea.

### Statistics

Comparisons between groups were performed using the Kruskal Wallis Test followed by post-hoc Dunn's-Bonferroni test. P<0.05 was considered statistically significant.

### Results

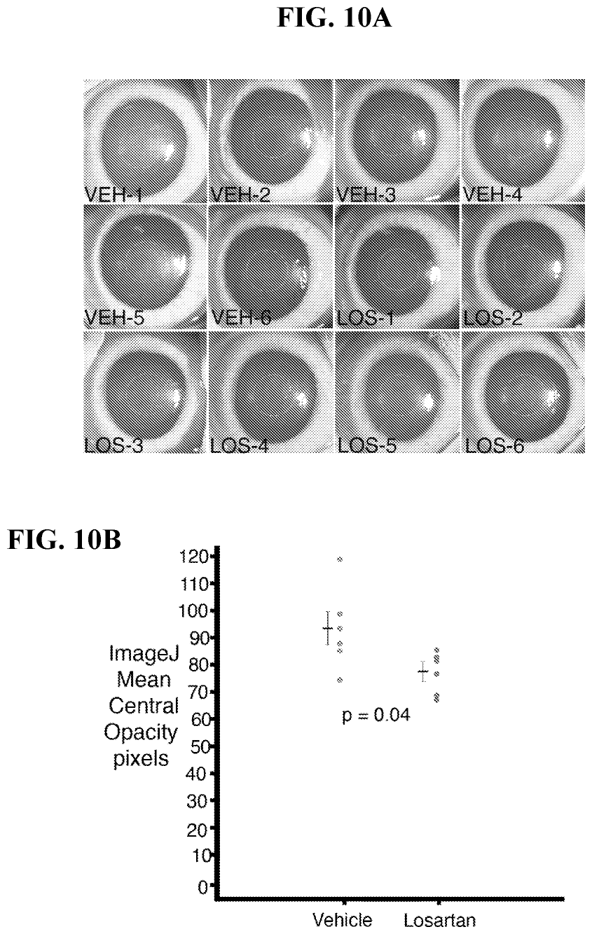

FIG. 10A provides standardized slit lamp photos from each cornea in the vehicle and losartan groups after −9D (on myopia scale) PRK and one month of topical drug treatment. This figure also shows the uniform central circular 3.5 mm diameter area within the PRK ablation in each cornea analyzed for opacity using ImageJ. FIG. 10B shows the mean pixels of opacity within the analyzed 3.5 mm diameter area for each cornea in the vehicle and losartan treatment groups. The difference between vehicle group (mean±SEM, 93±6 pixels) and the losartan group (mean±SEM, 77±3) was statistically significant (p=0.04).

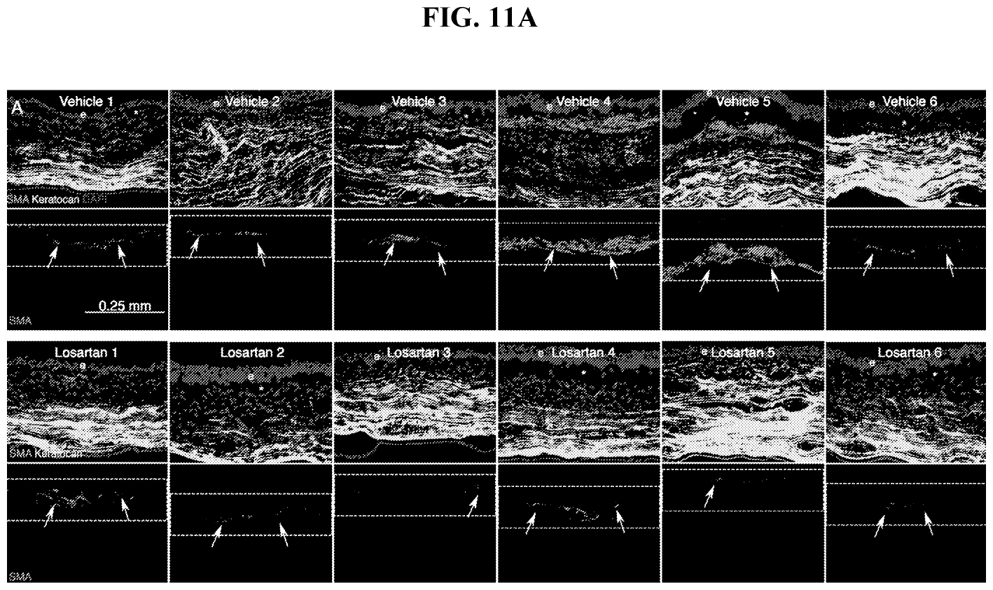

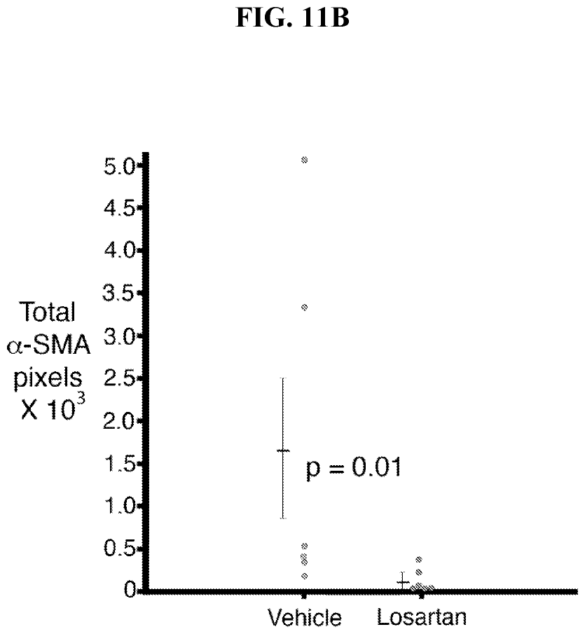

FIG. 11A shows representative duplex immunohistochemistry for α-SMA and keratocan in the central cornea in the vehicle and losartan groups after −9D PRK and one month of topical drug treatment. The 900-pixel wide by 235-pixel high area analyzed for total pixels of α-SMA staining intensity with ImageJ on the panel showing only α-SMA staining is also shown for each cornea. FIG. 11B shows a graph of the total pixels of α-SMA staining within the analyzed rectangle for each cornea. The difference between the vehicle-treated group (mean±SEM, 1630±840 pixels) and the losartan-treated group (mean±SEM, 120±60 pixels) was statistically significant (P=0.01). Note the much greater variability (higher SEM) in the vehicle group compared to the losartan group.

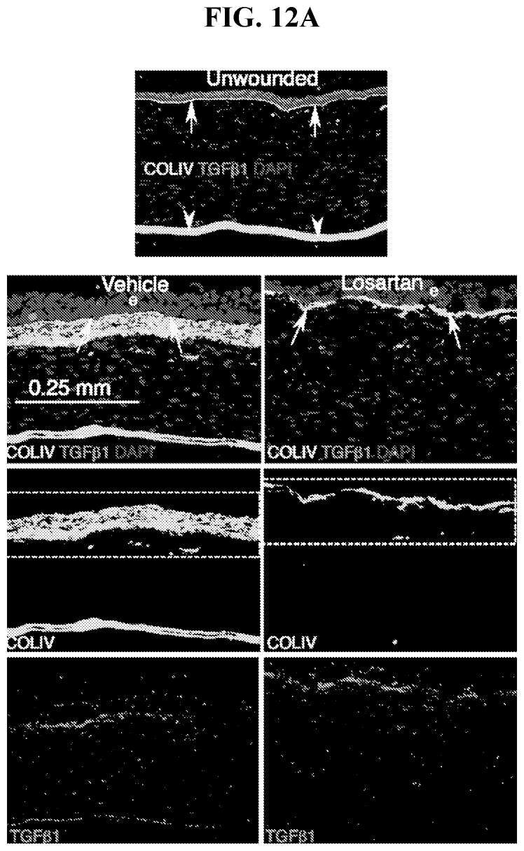

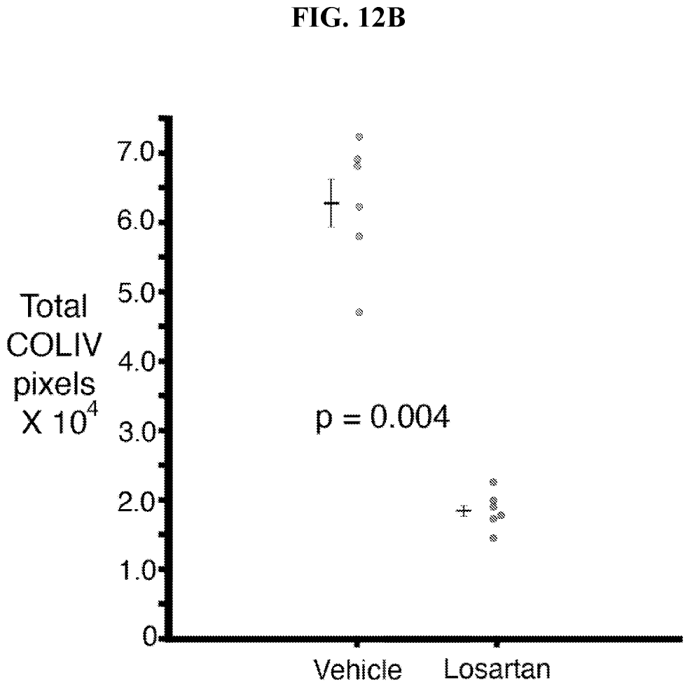

FIG. 12A shows representative example duplex immunohistochemistry for collagen type IV and TGF beta-1 in a representative unwounded control central cornea, as well as corneas in the vehicle and losartan treatment groups after −9D PRK and one month of topical drug treatment. In unwounded corneas, TGF beta-1 localized heavily to the epithelium and corneal endothelium, with smaller amounts detected in the stroma. In unwounded corneas, collagen type IV localized primarily to the epithelial basement membrane and Descemet's membrane, with little detected in the stroma. In corneas that had PRK and one month of vehicle or losartan treatment, TGF beta-1 was still localized to the epithelium and corneal endothelium, but also prominently to the epithelial basement membrane in both treatment groups. In corneas in the vehicle treatment group, a prominent line of TGF beta-1 was present in the stroma just beneath the collagen type I band in each cornea in that group.

FIG. 12A also shows that in the vehicle-treated group one month after −9D PRK, large levels of collagen type IV were present in a band in the subepithelial stroma beneath the epithelial basement membrane. In the losartan-treated group one month after −9D PRK, collagen type IV was localized primarily to the epithelial basement membrane, with far smaller amounts of collagen type IV detected in the underlying anterior stroma.

FIG. 12B is a graph of the total pixels of collagen type IV signal within the 900-pixel wide by 235-pixel high rectangle of stroma analyzed for each cornea. The difference between the vehicle-treated group (628700±37100) and the losartan-treated group (191000±9000) was statistically significant (p=0.004).

Late haze of the cornea after PRK is the clinical manifestation of stromal fibrosis resulting from the development and persistence of subepithelial myofibroblasts after surgery. These fibroblastic cells are themselves opaque due to their downregulation of corneal crystallins compared to keratocytes.²¹ Myofibroblasts develop from precursor cells that include local corneal fibroblasts (derived from keratocytes) and bone marrow-derived fibrocytes, which enter the cornea from the limbal blood vessels in response to corneal injury.^{22,23} Once mature myofibroblasts develop in the subepithelial stroma, they produce large quantities of disordered extracellular matrix components, such as collagen type I and collagen type III, that further compromise corneal transparency.²² The development of myofibroblast precursors into mature myofibroblasts, and the persistence of myofibroblasts in the tissue, is dependent on an ongoing and adequate supply of TGF beta-1 and/or TGF beta-2, as well as other growth factors, including platelet-derived growth factor.^{6,10}

Several studies have demonstrated that delayed or defective regeneration of the epithelial basement membrane (EBM), that is produced through the coordinated efforts of the epithelium and keratocytes/corneal fibroblasts, underlies the development of late haze fibrosis.^{6,10} Perlecan and collagen type IV are critical EBM components that modulate the entry of TGF beta-1 and TGF beta-2 into the stroma from the epithelium and tear film. The development of late haze fibrosis likely decreases vision after PRK through a combination of the stromal opacity, irregularity transmitted to the corneal surface, and regression of the refractive effect of PRK.²² Once late haze develops in the rabbit or the human cornea, it persists until the normal EBM is regenerated—a process that typically takes several months in rabbits and many months to years in humans after the development of late haze scarring fibrosis. In this example, one month was selected for the analyses because many studies have shown that is the peak of corneal late haze fibrosis after PRK in rabbits.^{5-7, 10} Regeneration of the normal mature EBM re-establishes the barrier function for epithelial and tear TGF beta-1 and TGF beta-2, lowers subepithelial stromal levels of these pro-fibrotic growth factors, and leads to apoptosis of the myofibroblasts²⁴ or possibly their reversion back to corneal fibroblast phenotype, although the latter mechanism of disappearance has yet to be demonstrated in situ. Once the myofibroblasts in the stroma decline, corneal fibroblasts and keratocytes re-enter the affected stromal tissue and reabsorb/reorganize the disordered extracellular matrix produced by the myofibroblasts to increase corneal transparency.²²

Each of the corneas at one month after −9D PRK and treatment with topical losartan in FIG. 10A had residual central opacity despite the decrease in myofibroblasts. This opacity is largely attributable to disordered extracellular matrix (ECM) components, such as collagen type I and collagen type III, produced by corneal fibroblasts and the few myofibroblasts that developed despite losartan treatment at this early time point after surgery.^{21,22} Many corneas that have PRK without the development of myofibroblasts develop clinically insignificant haze due to decreased crystallin production by corneal fibroblasts and the production of relatively low levels of disordered ECM by these cells.^{21, 22} This transient opacity typically decreases over a few months as the corneal fibroblasts that developed in response to surgery disappear by a combination of apoptosis and reversion back to the keratocyte phenotype.²² Thus, the corneas in both groups of this study would have likely become more transparent with longer follow-up.

This example demonstrated that topical losartan decreases non-basement membrane stromal collagen type IV production that is also up regulated in corneal fibroblasts via TGF beta signaling (FIG. 12A-B).^{19, 20, 27} Since the stromal collagen type IV appears to function to directly bind TGF beta-1 and TGF beta-2 to modulate interaction of the TGF beta with its receptors²⁷ there could be concern that inhibition of this collagen type IV production could have a detrimental effect on the overall TGF beta-mediated fibrosis response. That, however, has not been noted in the present example.

### REFERENCES FOR EXAMPLE 3

1. Lipshitz et al., Ophthalmology. 1997; 104:369-73
2. Raviv et al., J Cataract Refract Surg. 2000; 26:1105-6.
3. de Oliveira R et al., Exp Eye Res. 2020; 200:108218.
4. Kaiserman et al., Cornea. 2017; 36:961-966.
5. Torricelli et al., Invest Ophthalmol Vis Sci. 2013; 54:4026-33.
6. de Oliveira et al., J Refract Surg. 2022; 38:50-60.
7. Marino et al., J Refract Surg. 2017; 33:337-346.
8. Wilson et al., Corneal Invest Ophthalmol Vis Sci. 2020; 61:28.
10. de Oliveira et al., Exp Eye Res. 2021; 202:108325.
11. Wilson et al., Exp Eye Res. 2021; 207:108594.
12. Wilson S E. Cell Mol Life Sci. 2022; 79:144.
13. Wylie-Sears et al., Biochem Biophys Res Commun. 2014; 446: 870-5.
14. Geirsson et al., Circulation. 2012; 126(11 Suppl 1):5189-97.
15. Park et al., Cell Transplant. 2012; 21:2407-24.
16. Lim D-S, et al., Circulation. 2001; 103:789-91.
17. Lavoie et al., J Hypertens. 2005; 23:1895-1903.
18. Cohn et al., Nat Med. 2007; 13:204-10.
19. Sampaio et al., Exp Eye Res. 2022; 216:108940.
20. Sampaio L P, Hilgert G S L, Shiju T M, Santhiago M R, Wilson S E. Topical losartan and corticosteroid additively inhibit corneal stromal myofibroblast generation and scarring fibrosis after alkali burn injury. Trans Vis Sci Tech, in press.
21. Jester et al., J Cell Sci. 1999; 112:613-22.
22. Wilson et al., Invest. Ophth. Vis. Sci. 2022; 63:22.
23. Lassance et al., Exp. Eye Res. 2018; 170:177-187.
24. Wilson et al., Exp. Eye Res. 2007; 85:305-11.
25. Jester et al., Cornea. 1997; 16:177-87.
26. Angiotensin II Receptor Antagonists, In: LiverTox: Clinical and research information on drug-induced liver injury [Internet]. Bethesda (MD): National Institute of Diabetes and Digestive and Kidney Diseases; 2012-2017.
27. Wilson et al., Matrix Biol. 2022; 109:162-172.
28. Majmudar et al., Ophthalmology. 2000; 107:89-94.
29. Medeiros et al., Invest Ophthalmol Vis Sci. 2019; 60:1010-1020.
30. Sampaio et al., Exp Eye Res. 2021; 213:108803.
31. Waldrop et al., Cornea. 2020; 39:1227-1234.
32. Kobayashi et al., Ophthalmology. 2013; 120:923-927.
33. Abu el-Asrar et al., Eye (Lond). 1998; 12 (Pt 3a):453-60.
34. Schlunck et al., Exp Eye Res. 2016; 142:76-82.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

I claim:

1. A method of treating a subject with an eye condition comprising:
administering a composition to a cornea and/or conjunctiva of a subject, or providing said composition to said subject such that said subject administers said composition to said cornea and/or conjunctiva,
wherein said cornea or conjunctiva of said subject comprises an eye condition, and
wherein said composition comprises: a) a drug agent, wherein said drug agent comprises an ACE-2 receptor antagonist (also known as an Angiotensin II Receptor Blocker), and
wherein said eye condition is selected from: i) a corneal scarring fibrosis, ii) a transforming growth factor beta-induced (TGFBI) corneal dystrophy, iii) a conjunctival fibrotic disease, iv) an intraocular fibrotic disease, and v) a conjunctival bleb scarring and/or shunt encapsulation.

2. The method of claim 1, wherein said composition further comprises:
b) water, and
c) at least one of the following:
i) one or more salts present at a level such that said composition,
when in aqueous form, has about a physiological concentration of said one or more salts and about a physiological pH;
ii) one or more gelling agents present at a level such that said composition is in the form of a gel; and
iii) one or more ointment forming agents, present at a level such that said composition is in the form of an ointment;
d) optionally a preservative, and
e) optionally a soothing agent.

3. The method of claim 1, wherein said drug agent is present in said composition at a concentration of 0.05 mg/ml to 2.0 mg/ml or 0.01 to 0.4 mg/ml or 5 mg/ml to 100 mg/ml.

4. The method of claim 1, wherein corneal scarring fibrosis is selected from the group consisting of: Descemetorhexis without graft, DSAEK or DMEK graft displacement, Alkali or acid burns, Sulfur mustard, chemical weapon burn, Proliferative vitreoretinopathy (PRK) late haze, PRK breakthrough late haze after MMC treatment, LASIK complications scar, Persistent epithelial defect, Recurrent Herpes simplex, virus (HSV) keratitis, HSV endotheliitis with scarring, Herpes zoster ophthalmicus, Bacterial keratitis, Fungal keratitis, and Acanthamoeba keratitis, Corneal lacerations with excessive fibrosis.

5. The method of claim 2, wherein said composition is free, or detectably free, of any additional reagents besides said drug agent, said water, and said one or more salts.

6. The method of claim 2, wherein said composition comprises said soothing agent, and wherein said composition is free, or detectably free, of any additional reagents besides said drug agent, said water, said one or more salts, and said soothing agent.

7. The method of claim 2, wherein:
a) said composition further comprises said preservative, and wherein said composition is free, or detectably free, of any additional reagents besides said drug agent, said water, said one or more salts, and said preservative, or
b) said composition further comprises said preservative and said soothing agent, and wherein said composition is free, or detectably free, of any additional reagents besides said drug agent, said water, said one or more salts, said preservative, and said soothing agent.

8. The method of claim 7, wherein said preservative is selected from the group consisting of benzalkonium chloride, sodium chlorite, sodium perborate, purite, benzododecinium bromide, ethylenediaminetetraacetic acid (EDTA), chlorobutanol, thiomersal, disodium edetate, oxychloro complex (SOC), and boric acid.

9. The method of claim 2, wherein said soothing agent is present in said composition, and wherein said soothing agent is optionally selected from the group consisting of: carboxymethyl cellulose, polyvinyl alcohol, hydroxypropyl methylcellulose, hydroxypropyl cellulose, and hyaluronic acid.

10. The method of claim 1, wherein said composition is present in an eyedrop container.

11. The method of claim 2, wherein said composition comprises said one or more salts and is in a liquid form, and further is free or detectably free of said one or more gelling agents and said one or more ointment forming agents.

12. The method of claim 2, wherein said composition comprises said one or more gelling agents and/or said one or more ointment forming agents, and is in the form of a gel or an ointment, and wherein optionally said gelling agents are selected from the group consisting of: hypromellose, carbomer homopolymer, and carboxymethylcellulose, and wherein optionally said ointment forming agent is mineral oil, and/or petrolatum.

13. The method of claim 1, wherein said administering, or said administers, is at least four or six or eight times daily for at least one week.

14. The method of claim 1, wherein said administering, or said administers, is conducted about every half hour for at least 8 hours.

15. The method of claim 1, wherein said TGFBI corneal dystrophy is selected from the group consisting of a stromal deposit, a Reis-Bicklers corneal dystrophy, a Thiel-Behnke corneal dystrophy, a Lattice corneal dystrophy type 1, a Granular corneal dystrophy type 1 and/or type 2.

16. The method of claim 1, wherein said Conjunctival fibrotic disease is selected from the group consisting of: Trachoma, Stevens-Johnson syndrome, Ocular cicatricial, pemphigoid, and Ocular graft-vs-host disease.

17. The method of claim 1, wherein said drug agent is selected from the group consisting of: losartan, telmisartan, valsartan, olmesartan, candesartan, irbesartan, eprosartan, azilsartan, and losartan metabolite EXP3174.

18. The method of claim 2, wherein said one or more salts comprise one or more, or all, of the following:
i) about 0.64% sodium chloride,
ii) about 0.075% potassium chloride,
iii) about 0.048% calcium chloride dihydrate,
iv) about 0.03% magnesium chloride hexahydrate,
v) about 0.39% sodium acetate trihydrate, and/or
vi) about 0.17% sodium citrate dihydrate.

19. The method of claim 1, further comprising: administering a corticosteroid to said cornea of said subject, or providing said corticosteroid to said subject such that said subject administers said corticosteroid to said cornea, wherein said corticosteroid is present in said composition or present in a separate composition.

20. The method of claim 1, wherein said composition is present in a conjunctival reservoir, or other continuous delivery device, that slowly releases said composition over time into the tears of said subject, and optionally wherein said eye condition comprises PVR (proliferative vitreoretinopathy).

21. The method of claim 1, wherein said composition is present in a porous collagen therapeutic contact lens that releases said composition over time.

22. The method of claim 2, wherein said composition comprises said preservative.

23. The method of claim 1, wherein said administering, or said administers, is conducted at least daily for at least one week, or at least 2 weeks, or at least one month, and wherein said subject has myopia score of X diopters just prior to said administering or said administers, and is Y diopters at the end of said at least one week, said at least 2 weeks, or said at least one month, and wherein Y is at least 1 diopter lower than X.

24. The method of claim 1, wherein said subject is a human.

25. The method of claim 1, wherein said cornea of said subject comprises said eye condition, and wherein said eye condition is an eye injury has occurred 1, 3, 6, 12, 24, or 48 hours prior to said administering or said administers.

26. The method of claim 1, wherein said administering a composition to a cornea and/or conjunctiva of a subject comprises said subject administering said composition to their own cornea or conjunctiva.

27. The method of claim 1, wherein said cornea of said subject comprises said corneal injury, and wherein said injury was caused by trauma, a chemical burn, a microbial infection, or a surgery.

28. The method of claim 1, wherein said cornea of said subject comprises said corneal injury, and wherein said injury was caused by photorefractive keratectomy (PRK) or phototherapeutic keratectomy (PTK).

29. The method of claim 1, wherein said cornea injury has occurred within five or less days of said administering or said administers.

30. The method of claim 1, wherein said cornea injury has occurred within 24 hours or less of said administering or said administers.

31. The method of claim 1, wherein said drug agent comprises losartan.

32. A method comprising:
delivering a system to a subject with an eye condition,
wherein said eye condition is selected from: i) a corneal scarring fibrosis, ii) a transforming growth factor beta-induced (TGFBI) corneal dystrophy, iii) a conjunctival fibrotic disease, iv) an intraocular fibrotic disease, and v) a conjunctival bleb scarring and/or shunt encapsulation, and
wherein said system comprises:
a) an eye dropper container or a contact lens, and
b) a composition comprising: i) a drug agent, wherein said drug agent comprises an ACE-2 receptor antagonist.

33. The method of claim 32, wherein said composition further comprises:
ii) water, and
iii) at least one of the following:
A) one or more salts present at a level such that said composition, when in aqueous form, has about a physiological concentration of said one or more salts and about a physiological pH,
B) one or more gelling agents present at a level such that said composition is in the form of a gel; and
C) one or more ointment forming agents, present at a level such that said composition is in the form of an ointment;
iv) optionally a preservative, and
v) optionally a soothing agent.

34. The method of claim 32, wherein said drug agent is present in said composition at a concentration of 0.05 mg/ml to 2.0 mg/ml or about 0.01 to 0.9 mg/ml or about 10 mg/ml to about 100 mg/ml.

35. The method of claim 33, wherein said composition is free, or detectably free, of any additional reagents besides said drug agent, said water, and said one or more salts.

36. The method of claim 32, wherein said composition is present in said eye dropper container.

37. The method of claim 32, wherein said Comeal scarring fibrosis is selected from the group consisting of: Descemetorhexis without graft, DSAEK or DMEK graft displacement, Alkali or acid burns, Sulfur mustard, chemical weapon burn, Proliferative vitreoretinopathy (PRK) late haze, PRK breakthrough late haze after MMC treatment, LASIK complications scar, Persistent epithelial defect, Recurrent Herpes simplex, virus (HSV) keratitis, HSV endotheliitis with scarring, Herpes zoster ophthalmicus, Bacterial keratitis, Fungal keratitis, and Acanthamoeba keratitis, Corneal lacerations with excessive fibrosis.

38. The method of claim 33, wherein said composition comprises said soothing agent, and wherein said composition is free, or detectably free, of any additional reagents besides said drug agent, said water, said one or more salts, and said soothing agent.

39. The method of claim 38, wherein said composition further comprises said preservative and said soothing agent, and wherein said composition is free, or detectably free, of any additional reagents besides said drug agent, said water, said one or more salts, said preservative, and said soothing agent.

40. The method of claim 32, wherein said composition further comprises said preservative, and wherein said composition is free, or detectably free, of any additional reagents besides said drug agent, said water, said one or more salts, and said preservative.

41. The method of claim 32, wherein said TGFBI corneal dystrophy is selected from the group consisting of: a stromal deposit, a Reis-Bicklers corneal dystrophy, a Thiel-Behnke corneal dystrophy, a Lattice corneal dystrophy type 1, a Granular corneal dystrophy type 1 and/or type 2.

42. The method of claim 32, wherein said Conjunctival fibrotic disease is selected from the group consisting of: Trachoma, Stevens-Johnson syndrome, Ocular cicatricial, pemphigoid, and Ocular graft-vs-host disease.

43. The method of claim 32, wherein said eyedrop container is a single-use container.

44. The method of claim 33, wherein said composition comprises said at least one ointment forming agent and is in the form of an ointment.

45. The method of claim 32, wherein said composition is present in said contact lens.

46. The method of claim 33, wherein said composition comprises said preservative.

47. The method of claim 33, wherein said composition further comprises said soothing agent, and wherein said soothing agent is selected from the group consisting of: methylcellulose, hydroxypropyl methylcellulose, dextran, glycerin, carbomer, hyaluronic acid, phospholipids, saturated fatty acids, unsaturated fatty acids, triglycerides, benzalkonium chloride, and sodium ethylenediaminetetraacetic acid.

48. The method of claim 32, wherein said subject is a human subject.

49. The method of claim 32, wherein said cornea of said subject comprises said corneal injury, and wherein said corneal injury has occurred 1, 3, 6, 12, 24, 48 hours, or 5 days prior to said delivering.

50. The method of claim 32, wherein said eye condition was caused by trauma, a chemical burn, a microbial infection, or a surgery.

51. The method of claim 32, wherein said eye condition was caused by photorefractive keratectomy or phototherapeutic keratectomy.

52. The method of claim 33, wherein said composition comprises said one or more salts and is in a liquid form, and further is free or detectably free of said one or more gelling agents and said one or more ointment forming agents.

53. The method of claim 32, wherein said eye condition comprises a comel injury that has occurred within 24 hours or less of said delivering.

54. The method of claim 32, wherein said drug agent comprises losartan.

55. A composition comprising:
a) a drug agent, wherein said drug agent comprises an ACE-2 receptor antagonist, and wherein said drug agent is present in said composition at a concentration of 0.01-0.09 or 2.3-100 mg/ml;
b) water, and
c) at least one of the following:
i) one or more salts present at a level such that said composition, when in aqueous form, has about a physiological concentration of said one or more salts and about a physiological pH;
ii) one or more gelling agents present at a level such that said composition is in the form of a gel; and
iii) one or more ointment forming agents, present at a level such that said composition is in the form of an ointment;
d) optionally a preservative, and
e) optionally a soothing agent.

56. The composition of claim 55, wherein said composition comprises said one or more gelling agents and/or said one or more ointment forming agents, and is in the form of a gel or an ointment.

57. The composition of claim 56, wherein said gelling agents are selected from the group consisting of: hypromellose, carbomer homopolymer, and carboxymethylcellulose, and wherein optionally said ointment forming agent is mineral oil, and/or petrolatum.

58. The composition of claim 56, wherein said composition is in liquid form and is free, or detectably free, of any additional reagents besides said drug agent, said water, and said one or more salts.

59. The composition of claim 55, wherein said composition is in liquid form and comprises said soothing agent, and wherein said composition is free, or detectably free, of any additional reagents besides said drug agent, said water, said one or more salts, and said soothing agent.

60. The composition of claim 55, wherein said composition further comprises said preservative and said soothing agent, and wherein said composition is free, or detectably free, of any additional reagents besides said drug agent, said water, said one or more salts, said preservative, and said soothing agent.

61. The composition of claim 55, wherein said composition further comprises said preservative, and wherein said composition is free, or detectably free, of any additional reagents besides said drug agent, said water, said one or more salts, and said preservative.

62. The composition of claim 61, wherein said preservative is selected from the group consisting of: benzalkonium chloride, sodium chlorite, sodium perborate, purite, benzododecinium bromide, and boric acid.

63. The composition of claim 55, wherein said wherein said drug agent is present in said composition at a concentration of 2.3-100 mg/ml.

64. The composition of claim 55, wherein said composition comprises said one or more gelling agents and is in the form of a gel.

65. The composition of claim 55, wherein said composition comprises said preservative, and said preservative comprises an antibiotic.

66. The composition of claim 55, wherein said composition comprises said soothing agent, and wherein said soothing agent is selected from the group consisting of methylcellulose, hydroxypropyl methylcellulose, dextran, glycerin, carbomer, hyaluronic acid, phospholipids, saturated fatty acids, unsaturated fatty acids, triglycerides, benzalkonium chloride, and sodium ethylenediaminetetraacetic acid.

67. A system comprising:
a) composition comprising:
i) a drug agent, wherein said drug agent comprises an ACE-2 receptor antagonist present in said composition at a concentration of 0.01-0.09 or 2.3-100 mg/ml;
ii) water, and
iii) at least one of the following:
A) one or more salts present at a level such that said composition, when in aqueous form, has about a physiological concentration of said one or more salts and about a physiological pH;
B) one or more gelling agents present at a level such that said composition is in the form of a gel; and
C) one or more ointment forming agents, present at a level such that said composition is in the form of an ointment;
iv) optionally a preservative, and
v) optionally a soothing agent; and
b) an eye dropper container or a contact lens.

68. The system of claim 67, wherein said system comprises said eye dropper, and wherein said composition is present inside said eye dropper.

69. The system of claim 67, wherein said system comprises said contact lens, and wherein said composition is present inside of, or on the inner surface of, said contact lens.

70. The system of claim 67, wherein said drug agent is present in said composition at a concentration of 2.3-100 mg/ml.

71. The system of claim 67, wherein said drug agent is present in said composition at a concentration of 0.01-0.09.


SEQUENCE LISTING (1 sequence)

SEQ ID NO: 1 - protein, 19 aa, synthetic construct
LRLDGNEIKPPIPIDLVAC